(19) United States
(12) Patent Application Publication
(10) Pub. No.: US 2008/0154099 A1
(43) Pub. Date: Jun. 26, 2008

(54) Health monitoring system and method

(75) Inventors: Dan Aspel, Regina (CA); Robert Martens, Regina (CA); Colin McAllister, Regina (CA)

Correspondence Address: WELSH & KATZ, LTD, 120 S RIVERSIDE PLAZA, 22ND FLOOR, CHICAGO, IL, 60606, US

(73) Assignee: SASKATCHEWAN TELECOMMUNICATIONS, REGINA (CA)

(21) Appl. No.: 11/982,909

(22) Filed: Nov. 6, 2007

(30) Foreign Application Priority Data
Nov. 6, 2006 (CA) 2,567,275

(51) Int. Cl. A61B 5/00; G06Q 50/00

(52) U.S. Cl. 600/301; 705/2

(57) ABSTRACT

A system for ongoing collection, transmission, storage, analysis, and presentation of physiological and other personal data from individuals is provided. The information is collected through a communications network from sources such as physiological sensors, existing databases, keyboard/keypad/mouse input, interactive voice response (IVR) systems, and web interfaces. Storage of information is provided by secure network data servers. Analysis algorithms are applied to the information at multiple points within the system to generate reports and alerts that may be presented through various interfaces to authorized system users, including patients, medical doctors and other Caregivers. The system also analyses and parses data, generating queries to the user/patient to complement the analysis, providing alerts, reminders and both lifestyle and medical-related feedback.

### CROSS-REFERENCE TO RELATED APPLICATIONS

This utility patent application claims priority to Canadian patent application serial no. 2,567,275, filed on Nov. 6, 2006.

### TECHNICAL FIELD

The present invention relates generally to healthcare and medical telephony, and more specifically, to a system for and method of collecting and managing physiological and lifestyle information for use by individuals, familial and personal Caregivers, and medical professions in managing heath and wellness decisions.

### BACKGROUND

The cost of providing healthcare services in industrialized countries is enormous; often on the order of 10-15% of a country's gross nation product (GNP). In countries with public healthcare, these costs consume a large portion of tax revenues. In countries without public healthcare, individuals are either saddled with direct costs, or with the cost of buying health insurance. Regardless of how the system is financed, costs are high and as costs increase, difficulties with waiting times and accessibility to services are also growing.

Waiting times are so great that many patients are even resorting to “medical tourism”, that is, traveling to foreign countries for quicker access to medical treatment. This is despite the fact that the patient will not obtain proper follow up and monitoring when he returns home, and the fact that the foreign facilities and practitioners may not meet the same standards that the patient would see in his home country. Many patients feel that the quicker services outweigh the risks.

Also, many people live in countries with tremendous healthcare facilities, but they simply do not have the financial resources to access those facilities. The high cost of private medical care is creating a class divide between the rich and poor which results in many social problems.

In any event, the cost of providing healthcare services has been growing steadily for decades despite many efforts to find a remedy. Thus, any system and/or method which allows these costs to be reduced or avoided, or health services to be improved, would be highly desirable.

In an effort to control medical costs, many healthcare systems attempt to remove patients from the hospital or other facility as quickly as possible, returning patients to their homes or otherwise placing them in the hands of non-professional Caregivers. These outpatient and home healthcare programs do seem to reduce direct costs, such as the cost of hospital beds, but many of these patients are sent home without any regular monitoring. Healthcare providers only receive patient data and feedback when the patient returns for an appointment at some time in the future. This time delay can aggravate healthcare costs if the patient's condition has deteriorated during their stay away from healthcare facility. The returning patient may, for example, require more costly and complex treatment than if they had stayed in the facility from the beginning.

Recent technological developments have allowed healthcare providers to monitor patients remotely and in many cases automatically. This has made outpatient programs more effective, particularly in the case of chronically ill patients who must be treated or monitored on a continuous or daily basis. More importantly this technology has contributed greatly to the quality of life for persons with these chronic illnesses through the reduction of co morbid conditions, hospitalizations and general peace of mind for patients and their loved ones.

Existing monitoring systems do not integrate multiple disparate devices together in an effective way, making the implementation of multiple devices expensive, complex and prone to error. Multiple separate systems have to be purchased and operated, but more importantly, they must be monitored by an individual who can analyze the collective significance of the data. Clearly, it is impractical to have an individual monitoring these disparate devices on a continuous basis, so it is simply not done.

For example, devices and systems exist to monitor certain patient data such as blood pressure and temperature. However, these systems are typically provided as separate dedicated devices with a single use, and they cannot be adapted to provide data on any other patient conditions or information. The healthcare provider may simply receive blood pressure or temperature data without any other information regarding the context—information which might be necessary for the device data to be of any use at all. If the healthcare provider wishes to receive a number of kinds of patient data, such as heart rate, blood pressure, temperature and heart valve signal, then he will likely have to purchase, setup and monitor four completely independent systems. When data is received, it will not be synchronized, correlated, arrive in the same format or even on compatible software systems. Thus, the healthcare provider will have to perform considerable manipulation and analysis before he can make any determinations from the data.

If an effective remote health monitoring and management system could be developed, the frequency and cost of follow-up appointments and testing could be reduced. This would save both the patients and the healthcare providers time and convenience, as well as reducing the resources required. Healthcare performance would also improve, as patients could be contacted before a major crisis ensues. Furthermore, the patients, along with their family and friends, would feel more confident with the patient's condition being continuously and safely monitored.

There is therefore a need for an improved health monitoring system and method, with regard to the problems outlined above.

### SUMMARY

It is an object of the invention to provide an improved health monitoring and management system and method.

Existing healthcare telemonitoring and management systems are uni-directional, simply extracting data from the patient and providing it to the healthcare provider. There is currently no feedback loop between the client and the Caregiver—be it a patient and healthcare provider relationship, a mother and son relationship or an individual wanting to see their own information in a meaningful format. Closing the feedback loop between the client and the Caregiver improves the efficiency and effectiveness of providing healthcare services: increased quality and length of life, decreased travel and hospital time, reduced comorbidities associated with chronic and acute illnesses and lifestyle concerns for patients/clients. It also provides professional Caregivers with the information they require to properly manage their clients' illnesses without actually having to see the patient in person. Specialists from around the globe are able to assess the same data in real time thus overcoming the geographical boundaries that exist today. Many regions do not have access to specialists and as such the patients are put on long waiting lists and then have to travel long distances to access care. This burden is drastically reduced by the system of the invention. This is true in the treatment or monitoring of chronic and acute illness. For the loved one, it creates a sense of ease knowing that their loved one has taken their vitals and they are acceptable. For the consumer it provides a tool to help them better manage their health and fitness.

There is currently no universal standard for communication devices, be they wireless or hardwired. Each device uses it own standard and the mobile devices do not talk to one another, or to fixed devices. The disclosed platform provides a means for easily accommodating such disparate devices and integrates them together with a management system.

In addition, the transmission of further queries to the patient in response to certain data being received is provided. This allows a truly comprehensive analysis to be performed. None of the existing systems provide such functionality. The parsing of data, analysis and generation of queries can be effected using an expert system, a rules engine, artificial intelligence or be hard-coded. Other systems may also be used. These systems all accommodate the analysis and synthesis of data from various disparate inputs, which has not been available in the past.

Wireless technologies such as Bluetooth™ (or other short range wireless radio), CDMA (Code Division Multiple Access), satellite and GSM (Ground System for Mobile) are leveraged to allow for a truly wireless solution while the system also has the functionality to use traditional PSTN (Public Switched Telephone Network) line and IP (Internet Protocol) technologies. The system is designed with patient centricity in mind and as such focuses on closing the feedback loop between the client (patient) and Caregiver (professional or loved one). As shown in FIG. 5, data readings from various medical devices are received by a local access point, and transmitted to a central database. The data is processed and feedback provided to the user.

This is achieved through real-time, and store and forward delivery of desired information via web interface, automated interactive voice response, SMS text message (Short Message Service), fax, email, and voice mail in a meaningful format as well as directly through a customized user interface. The solution utilizes Canadian Medical Devices Conformity Assessment System (CMDCAS) approved third-party physiological data collection devices and transmits this information via Bluetooth (or other short range wireless radio) using software algorithms that ensure all data is accurately and securely collected from the point of origin as governed by applicable health regulations (PHIPA, HIPA, HIPAA, PIPEDA or whichever regulations are applicable in the jurisdiction of implementation) and delivered to the required destination.

The solution achieves this by connecting a Bluetooth radio (or other short range wireless radio) to the data collection device where one is not already integrated into the data collection device to gather data from the medical (or fitness equipment) device.

This requires specific code to be created for each device to enable the device to be supported by the communications system. Once the devices are configured so that they can communicate with one another the information is transmitted to the communication device—this may be a cellular telephone, a Bluetooth (or other short range wireless radio)/analog modem or a Bluetooth (or other short range wireless radio) enabled PDA (personal digital assistant) or PC (personal computer). The data is then analyzed, parsed and run through a series of queries (or expert system or the like) to determine the next action. Depending on the data, a question or series of questions may appear on the user interface or an interactive voice response (IVR) system may contact the client and provide information regarding their submission and ask pertinent questions as decided by the Caregiver. Data is forwarded via networks such as CDMA network, GSM network, satellite network, IP backbone or PSTN system to a secure data center. Should the network become unavailable all information may be stored at the point of transmission until the network becomes available again. The device will preferably attempt to resend the data at predefined intervals until successful or the user can initiate a resend of the data.

Patient physiological data such as blood pressure, blood sugar levels, weight, and oxygen saturation, is collected and transmitted to a secure central storage server which can be accessed by healthcare professionals for analysis and intervention. This data is also available to the patient for viewing purposes and to aid in self-management of their specific health condition.

The system incorporates an application service provider (ASP) model to facilitate a tele-health business. The interoperable design of this application will include the use of HL7 (standards for electronic interchange of clinical, financial, and administrative information among healthcare oriented computer systems).

The system allows both patients and/or the healthcare professionals to populate the central databases.

With respect to patient data, the system is designed in such a way that all data is completely anonymous and is only resolved when securely accessed by an authorized user. The entire system is compliant with all applicable health security standards.

This summary of the invention does not necessarily describe all features of the invention.

Other aspects and features will become apparent to those ordinarily skilled in the art upon review of the following description of specific embodiment of the invention in conjunction with the accompanying figures.

### BRIEF DESCRIPTION OF THE DRAWINGS

Further features and advantages of the present invention will become apparent from the following detailed description, taken in combination with the appended drawings, in which:

FIG. 1 presents a process flow diagram of the data transfer from a remote device, through the server system, and back to the user in an embodiment of the invention;

FIG. 2 presents a process flow diagram of the data transfer from a remote device through to the data centre, via a landline access point, in an embodiment of the invention;

FIG. 3 presents a process flow diagram of the data transfer from a remote device through to the data centre, via a wireless cellular network, in an embodiment of the invention;

FIG. 4 presents a block diagram of the system architecture in an embodiment of the invention;

FIG. 5 presents a block diagram of the overall system architecture in an embodiment of the invention;

FIG. 6 presents a flow chart of a method of operation for the system in an embodiment of the invention;

FIG. 7 presents a flow chart of a method of operation of a landline access point in an embodiment of the invention;

FIG. 8 presents a flow chart of a method of operation of a cellphone in an embodiment of the invention;

FIG. 9 presents a flow chart of a method of operation of an application server in an embodiment of the invention; and

FIG. 10 presents a block diagram of an alerting engine in an embodiment of the invention;

FIG. 11 presents a block diagram of web interface structure of system level use cases in an embodiment of the invention;

FIG. 12 presents a block diagram of the web interface structure of summary pages view of use cases in an embodiment of the invention;

FIG. 13 presents a block diagram of the web interface structure for specifying reporting criteria in an embodiment of the invention; and

FIGS. 14 through 36 present screen captures of various user interfaces, announcements and reports in an embodiment of the invention.

It will be noted that throughout the appended drawings, like features are identified by like reference numerals.

### DETAILED DESCRIPTION

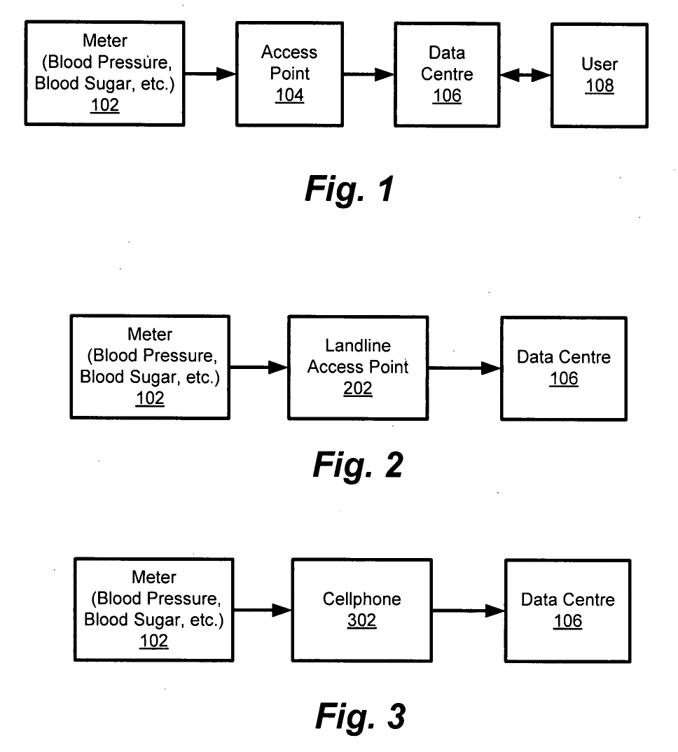

Embodiments are described below, by way of example only, with reference to FIGS. 1-36. The present invention will be presented by means of the following examples.

Collection, transmission, and storage of physiological and lifestyle data originating from patients is a necessary requirement of an effective automated telemonitoring system. The described system has the necessary communication protocols to enable the patient to use home medical monitoring devices such as a blood pressure monitor, a glucometer, and all other devices capable of collecting physiological and lifestyle data for transmission to the data center. Readings are taken in the same fashion as any patient currently using these devices would. Data readings are retained within the medical devices as per manufacturer's specifications without regard to the described system.

System Operation

FIG. 1 presents a process flow diagram of the system at a high level. In its essence, the system collects data from medical and measurement devices 102 via an access point 104 that is local to the patient and devices 102. The access point 104 in turn, transmits the data to a data center 106 which securely stores that information, analyses it and provides interfaces for various users 106 to receive guidance, view and interact with that data.

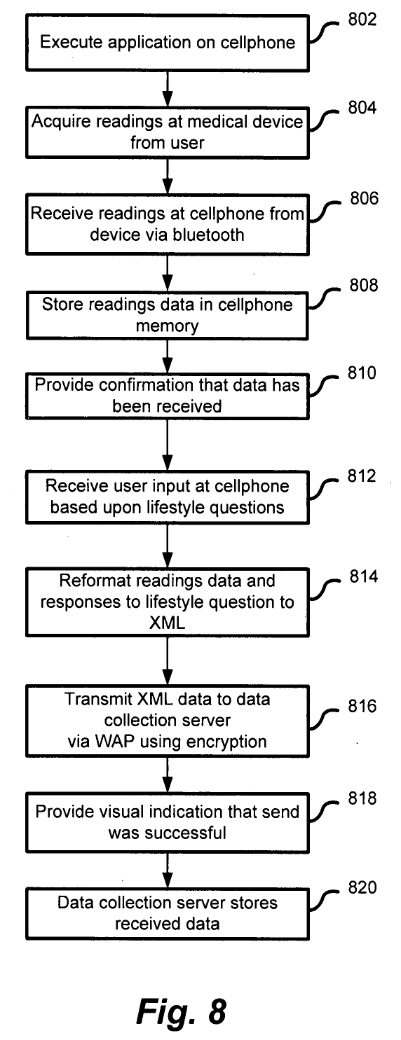

FIG. 2 presents a process flow diagram of the data transfer from a remote device 102 through to the data centre 106, via a landline access point 202, the method of which is described in connection with FIG. 8.

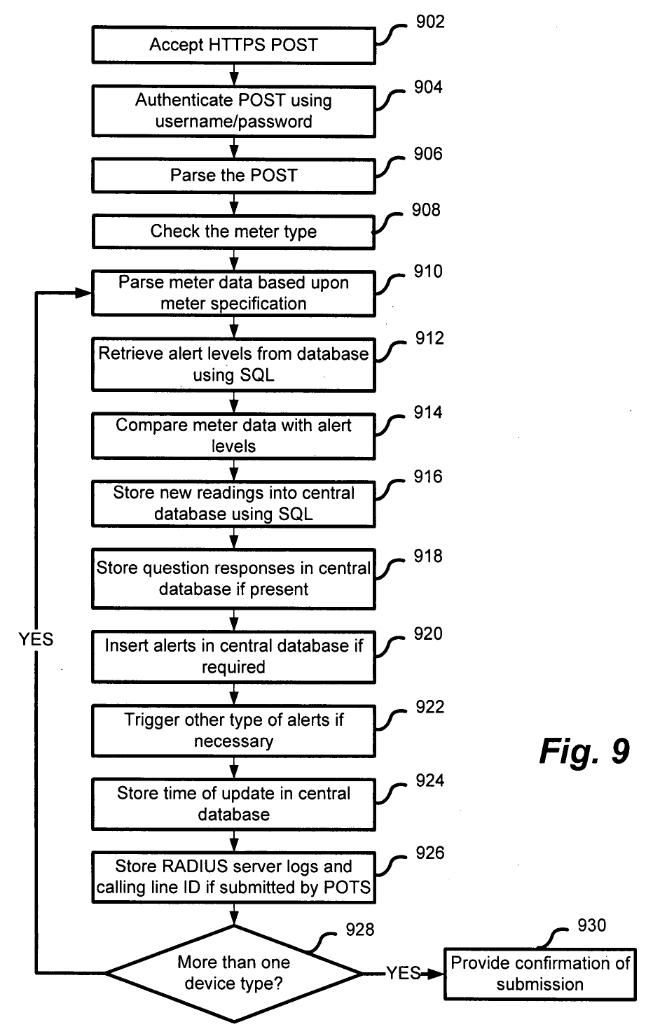

FIG. 3 presents a process flow diagram of the data transfer from a remote device 102 through to the data centre 106, via a wireless cellular network by a mobile device such as a cellphone 302, the method of which is described in connection with FIG. 9.

Detailed System Architecture

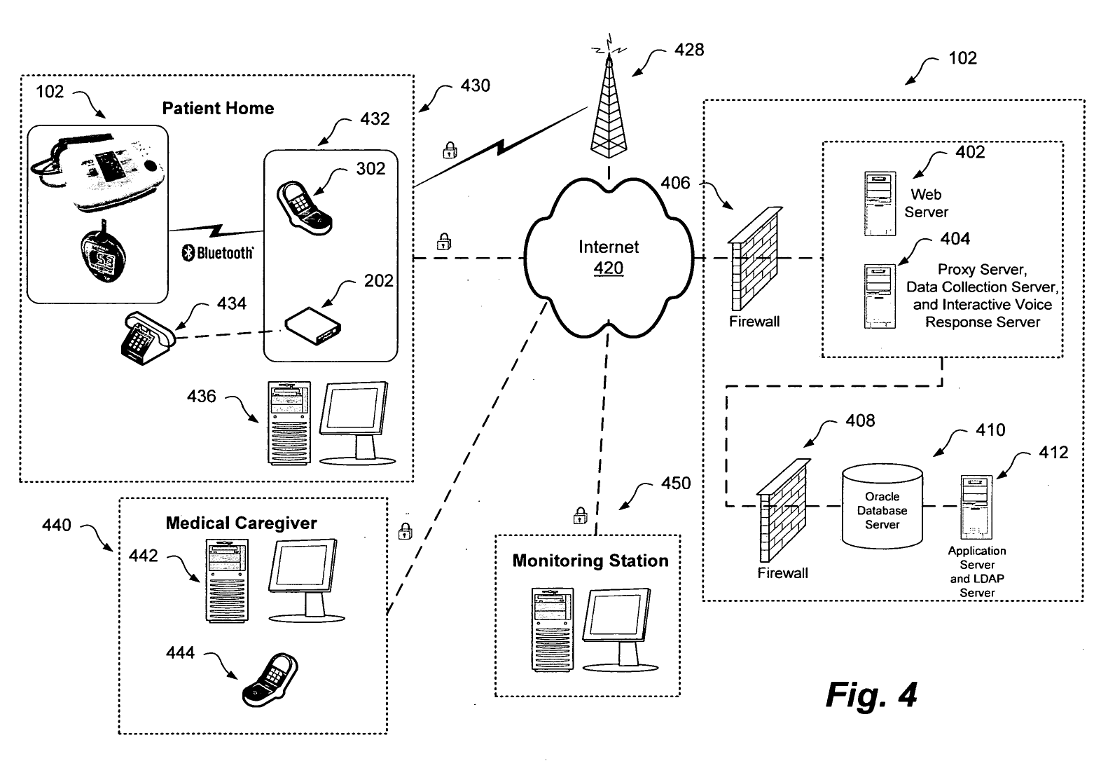

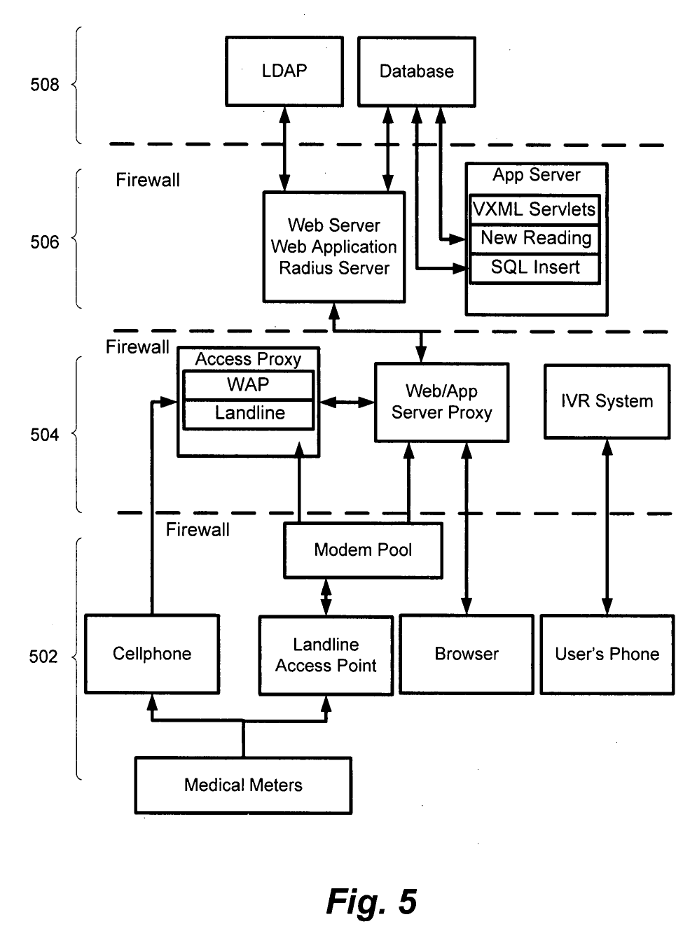

FIGS. 4 and 5 presents a much more detailed block diagram of the system architecture.

FIG. 4 shows how the various devices and their interconnectivity could be implemented, dividing these components up into patient home, central system, monitoring station, and medical Caregiver locations. A data centre 102 is designed with PIPEDA and HIPPA privacy regulations using SSL (Secure Socket Layer) handshake and encryption. The internet 420 is connected to the data centre 102 by a firewall 406 to provide access for a web server 402 for generating web pages for collection and presenting patient data provided through proxy server, data collection server, and interactive voice response server 404. Data can then be further secured behind a firewall 408 on an Oracle database server 410 and application server and LDAP server 412 to protect patient data. The data can be protected utilizing the Health Level 7 ANSI standard for healthcare specific data exchange.

The patient's home 430 can be connected to the data center by multiple communication means provided by access devices 432. Whatever data is submitted, a patient will preferably be prompted for activity-related information through their cellular phone user interface or an IVR system for landline users. Wireless access can be provided through a cellular network 428 using SSL encryption for sending and receiving data to wireless devices such as cellphone 302. Alternatively, an access point 202 can be utilized to provide voice access by a telephone 434 utilizing a dial-up data communication AES Encryption 128 bit to send meter data and receive configuration data. Data is transferred from the medical devices. A patient may also be contacted directly by a monitoring agent when the agent notices an alert event. If the event requires additional medical attention the patient will preferably be contacted by their medical Caregiver. A personal computer 436 may also be utilized to provide data entry access through a secured internet connection.

A monitoring station 450, connected to the data centre 102, is utilized by monitoring agents to watch incoming patient data. When an alert event is noted by a monitoring agent the patient will preferably be contacted by the agent. If the patient is unavailable or is in need of medical attention the patients medical Caregiver will be contacted by the monitoring agent.

A medical Caregiver 440 may access the patient data through the data centre 102 by a personal computer 442 or a wireless device 444 such as a cellphone or smartphone device. The medical Caregiver is contacted by a monitoring agent whenever medical attention is required by the patient.

The system is based on a layered architecture as presented in FIG. 5. This architecture provides multiple layers of security for the data stored in the database and LDAP (lightweight directory access protocol). LDAP is a set of protocols for accessing information directories, which supports TCP/IP, thereby supporting Internet access.

The first layer 502 consists of medical devices, access devices and a modem pool with a toll free number. This layer allows:

1. the user's medical devices to transmit data using a cellular telephone or landline access point and modem;
2. the user to view data and information stored on the system via a computing device (such as a PC) and Web browser; and
3. the user to communicate with the IVR (interactive voice response) system via his local telephone.

The entire layer is preferably protected with a firewall.

Note that the modem pool is the only module in the first layer, that is in the central system location rather than at the user's location.

The second layer 504 proxies traffic to the appropriate software applications in the third layer. This layer performs any data format translations necessary, handles terminations of cellular traffic, and hosts the IVR system that is used to interact with the user. The second layer is isolated from the first and third layers with a firewall.

The third layer 506 holds the main logic of the system. It controls access to the information stored in the LDAP and Central Database of the fourth layer, inserts data into the Central Database, and provides presentation services for content in the Central Database.

The deepest layer 508 contains the LDAP and database. The LDAP contains identifiable user information and the database contains the user's medical data. The information is separated from the third layer via a firewall, for additional security and for internal purposes to limit the visibility of information to system administrators.

Web Application

The Web application as shown in FIG. 8, is one of the user interfaces to access data stored in the system. The Web interface allows authorized users to add and delete users, view data and delegate access to data based on user roles.

The Web application provides access to lifestyle, physiological, and medical data stored in the system. It provides raw data views, traditional data views, and reports (text and graphical) based on automated and manual analysis of the data. Raw data views show the user raw data that was submitted in the greatest detail. This allows the user to find out exact details such as the time that the reading was taken. Traditional views of the data mimic the ways patients and medical professionals are currently trained to view data such as a log book. Finally, the system can provide reports that analyze data so patients can get a clear view of their current medical state without the need to pour through tables that show individual readings.

The web application is designed for the patient to view their data along with a number of other persons simultaneously. The persons who are able to view the data in addition to the patient are configurable within the web application.

The web application has a multi-tiered administration tool that supports roles for doctors and other users to create users and suspend other users. This allows for the use of flexible billing and provisioning models. In particular, administrators can activate users that would be billed individually while doctors could activate users that are billed as a whole to either private or public health insurance.

Central Database

The central database stores the user's physiological and medical readings, answers to lifestyle questions, alerts, and information about submitted readings. The central database does not store identifiable information to improve security. Instead, each user's data is linked to a unique account ID.

The central database could be implemented as an SQL database such as Oracle. It also uses redundancy and backups to ensure integrity and safety of medical data in the case of failures and provide methods for disaster recovery.

LDAP

A lightweight directory access protocol (LDAP) server (such as open LDAP) is used to store user information. This keeps identifiable patient information separate from the medical data in the database for increased security. The LDAP server also stores log-in, user authentication, and rights information.

Modem Shelf

A dedicated modem pool with a toll free number is used to accept data from landline access points.

The modem shelf is protected by its own log in credentials so that only acceptable client devices can log into the modem shelf. Authentication and accounting information for landline data submission is sent to a standard customer RADIUS server.

Additionally, the modem shelf is configured to send accounting information, including “Calling-Station-Id” to a dedicated RADIUS server. This provides logging of where data is being submitted from and provides the IVR subsystem with the information necessary to call users back with lifestyle questions after a medical reading is submitted.

RADIUS Server Proprietary Application

The Remote Authentication Dial In User Service (RADIUS) is an MA (authentication, authorization and accounting) protocol for applications such as network access or IP mobility. The RADIUS server logs accounting packets from the modem pool (see the third layer of Figure H). A proprietary application running on the same server correlates user ID for submitted readings with “Calling-Station-Id” based on a timestamp and IP address. This information is then placed in the database so that it is accessible to the IVR system.

The RADIUS Server also logs information about data submitted by the POTS (plain old telephone system) accounting packets are sent to secondary (LifeStat) RADIUS server.

Authentication and accounting information for landline data submission is also sent to a customer RADIUS server for the modem pool.

Finally, a server along side the secondary RADIUS server accepts requests for: “Calling-Station-Id” based on a timestamp and IP address from the data collection server. The server responds with “Calling-Station-Id” and time difference from matching timestamp. If the time difference is within a few seconds than the “Calling-Station-Id” is known to correlate with the IP address

Interactive Voice Response System (IVR)

If the patient is using a landline (PSTN) based system the data will automatically be transferred to the data center without any additional patient input. If the healthcare professional requires additional lifestyle information such as when a reading was taken relative to a meal, etc., then the patient will be phoned immediately subsequent to taking their readings by an automated multi-lingual voice prompted IVR system running proprietary Voice XML scripts. This IVR will indicate to the patient that their readings were successfully received and have them answer pertinent questions with respect to their readings. The user may input his answers by selecting a number on the dial pad of their phone. If there is a transmission failure using the landline based solution the patient will not be contacted by the IVR. However, the readings will be retained within the access point located in the patient's home until a successful transmission is made.

The interactive voice response system (IVR) calls patients to ask them lifestyle questions about readings they submit. It receives lifestyle information from the user for readings that require lifestyle questions to be answered but that were not answered prior to submission because neither the access point nor the medical device had the ability to ask the questions and receive input. Since the IVR system continually scans the database, calls will be made to the user as soon as the reading is received. As a result, the user experiences a seamless process of taking their reading and answering the lifestyle question.

The central database is polled for new ‘pending’ telephone numbers (which is associated with a specific patient) at select time intervals or after the last attempted outbound call has been completed (which ever is sooner) using a Java servlet that resides on a Java application server. This servlet then provides the necessary information to the IVR server to generate an outbound call to a patient who has readings that require additional information to be completed.

If there is no answer/busy/hang up before completion, the call will be attempted a set number of times with a set delay between call attempts. After a set number of unsuccessful attempts the patient will be removed from the pending group. However, those readings can still be updated once additional readings with missing data have been submitted as the IVR is prompting for all outstanding incomplete readings when it is interacting with a patient.

The questions the IVR asks a patient is dynamically generated using Java JSP's to generate VXML specific to that patient and their information in the database.

The IVR receives answers in the form of DTMF (dual tone multi-frequency) tones from key presses on the user's telephone, interprets them and updates the central database.

Alerting

The system is designed to alert users and Caregivers based on the reception of certain data or the absence of data within a set time. FIG. 1 presents a block diagram of how the alerting engine interacts with the rest of the system.

Alerts can be generated when

1. readings are out of a configurable set of target ranges within a defined period of time;
2. a reading has not been received with a set number of days;
3. equipment is reporting error or low battery conditions;
4. a number of user defined alerts are detected; or
5. if user has reconfigured time on peripheral device.

Alerts can be presented to patients, their Caregivers, designated medical professionals and monitoring centers in the web interface to the system. Once logged in, the user may see all alerts pertaining to them and persons within their care. These alerts can be sorted by date, importance and person.

Alerts can be delivered by all previously mentioned voice and data delivery systems. This allows the user to be informed of the alerts, to acknowledge alerts, and to enter additional information in response to the alerts. Alternatively, alerts can be delivered via short message service (SMS) message to a user's cellular telephone.

Alerts can be delivered to a monitoring centre. The alert information can then be viewed along with the patient information to determine a course of action which could, for example, be a telephone call to that person to either check their status or provide education on how to better manage their condition.

Alerts may also be delivered via e-mail, fax, phone, SMS text message or other user desired communication protocols.

Submitting Readings with a Landline Access Point

The functionality of this device will depend to a large extent on how the system designers and/or administrators wish to operate their system. Exemplary functionality is described hereinafter but it is straightforward to modify or add to the system based on this description. This functionality can easily be provided using a microcontroller, microprocessor, digital signal processor or ASIC (application specific integrated circuit) of some kind, volatile and non-volatile memory components and appropriate interface hardware and software. A typical device will be required to receive readings via a Bluetooth communication channel, store received readings, confirm receipt of readings and communications, if readings are in storage then connect to the modem pool, connect to the server via an IP (Internet Protocol) connection, send readings, wait for acknowledgements, delete readings when a positive acknowledgement is received, sleep until a new reading or a retransmit timeout is received, a negative acknowledgement or inability to connect.

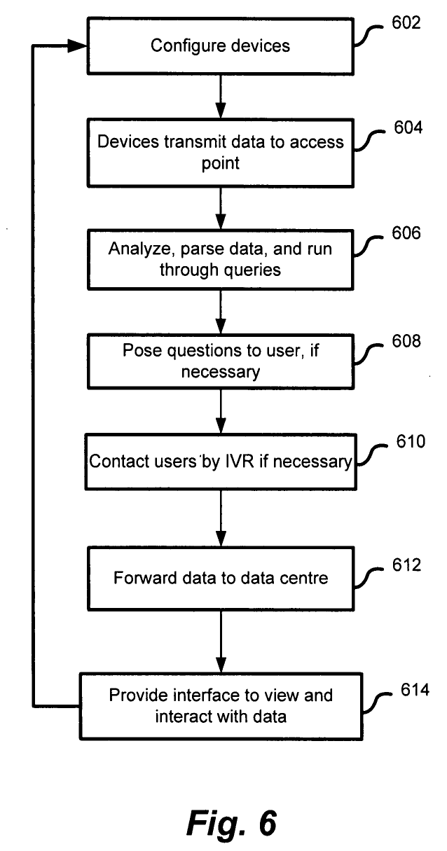

FIG. 6 shows a high-level method of operation of the system in which:

1) The medical devices are configured at step 602;
2) Devices transmit data to the access point at step 604;
3) Data is analyzed, parsed and run through queries at step 606;
4) Lifestyle questions are posed to the user, if necessary, based upon the data transmitted from the device at step 608;
5) The user is contacted by IVR (interactive voice response), if necessary, to respond to lifestyle questions if electronic access is not available at step 610; and
6) The data is then analyzed and accessible by a web interface to enable interaction at step 614 by a care provided.

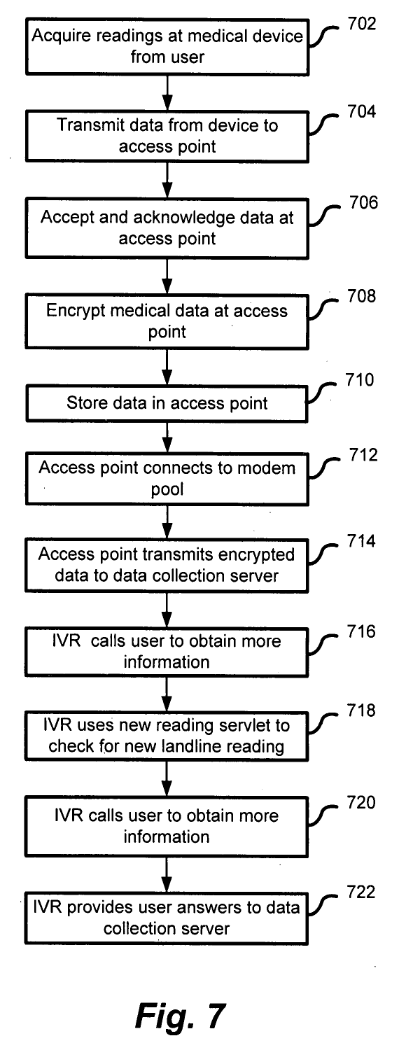

The landline access point receives data from medical devices and transmits the data over a PSTN telephone line to the data center where the data is stored, as shown in the block diagram of FIG. 4. The process generally proceeds as shown in FIG. 7.

The process begins when the User takes a physiological reading using a medical device at step 702.

The reading from the medical device is transmitted via Bluetooth or some other short range wireless radio, to a landline access point at step 704.

The landline access point accepts and acknowledges the medical data at step 706.

The medical data is encrypted with AES (advanced encryption standard) or some similar encryption algorithm at step 708. AES is widely used and is the current standard for the U.S. Government. It is a 128-bit symmetric block encryption technique.

The medical data is stored in the access point in non-volatile memory in case re-transmission is required at step 710. Once confirmation of successful transmission is received the data is deleted.

The access point connects to the modem pool using appropriate credentials at step 712.

The access point transmits the AES encrypted medical data to the landline proxy server in the data center at step 714. If transmission fails, the access point retransmits the AES encrypted medical data at predefined intervals or the next time a reading is received.

The landline service decrypts the AES encrypted medical data and then reformats the data into the data reception and SQL insert service's XML specification at step 716. The data are then sent to the data collection servlet using a SSL (secure sockets layer) HTTPS POST to the data collection servlet on the Application Server in the data center. The landline service waits for an accepted/rejected message from the servlet which is then sent as a positive or negative confirmation to the landline access point.

The data collection servlet parses the reading(s), generates specific alerts based on the reading and prior readings, queries an application running on the RADIUS server for a telephone number, and stores the reading(s), alerts, and telephone number into the database at step 718. More information on the data collection servlet is described in the Application Server—Data Reception and SQL Insert section.

The IVR uses the New Reading servlet to check for new landline readings. If a new reading is detected, the caller ID information is retrieved and the client is called at that number at step 718. More information about how the caller ID information is obtained is described in the RADIUS Server section.

Once a call is established, the IVR calls the VXML servlet which generates a voice XML call flow based on the readings in the database that need to have lifestyle questions answered at step 720. More information about how the IVR calls the user and obtains lifestyle information is provided in the IVR section.

The IVR then calls a servlet to insert the answers to the lifestyle questions into the database at step 722.

Submitting Readings with a Cellular Phone Access Point

As an alternative to the landline access point, medical device readings may be received and forwarded to the central database via a Bluetooth-enabled cellular telephone as presented in the block diagram of FIG. 4. Many existing cellular telephones already have hardware support for such functionality. The process generally proceeds as shown in Figure.

This process is initiated when a dedicated software application is started on the cellular telephone by the user at step 802. On cellular telephones that support automatically starting a software application, this step can be skipped as the software application can be started by a Bluetooth (or other short range wireless radio) transmission from the user's medical device. The software application may be downloaded wirelessly and installed by the cellular user using the web browser on the phone.

The User then takes a physiological reading. This could be a point reading or a continuous reading.

The reading is transmitted via Bluetooth (or other short range wireless radio) to the cellular telephone at step 804.

The cellular telephone stores the reading in non-volatile memory to ensure the reading is not lost at step 806. This is typically required because a network connection cannot be guaranteed on a cellular telephone. The reading is deleted once positive confirmation of transmission is received.

The cellular telephone notifies user that the reading has been received to provide feedback to the user that the data was successfully received at step 808.

The cellular telephone asks any lifestyle questions that are related to the nature of the data received at step 812. Lifestyle questions can be defined per user and per reading type. Also, questions can be asked based on the content of the readings.

The cellular telephone reformats the medical reading data and lifestyle questions into the dedicated XML specification at step 814. The cellular telephone then transmits the medical reading to the data collection servlet (SQL insert) using the WAP gateway and web/app server proxy in the data centre (using 128 bit SSL encryption) at step 816. If the transmission fails, the reading is stored and the Cellular telephone retries at prescribed intervals or when the user initiates a retry by taking another reading.

The cellular telephone provides a visual indication to the user that a medical reading is being transmitted and provides an indication of how many medical readings have been transmitted out of the total number of readings to be transmitted at step 818. This allows the user to know when the application on the cellular telephone can be shut down. A user would normally wait until all readings were transmitted but if the user needs to use the telephone, they could terminate the software application and know that they would need to restart it later to transmit the remaining readings.

The data collection servlet stores the medical reading and answers to the lifestyle questions into the database at step 820.

Although is it preferable to include all of the intelligence and processing described above on the cellular telephone, the intelligence could be left on the central system. In such an embodiment the cellular telephone would operate simply as a communication channel in much the same manner as the landline access point.

The software application on the Cellular telephone could be provisioned in several ways, including the following:

1. The user provisions the cellular telephone application themselves though the application could be preloaded on the phone. This allows the application to be deployed to any compatible cellular telephone even if the cellular telephone is on a different network.
2. The cellular telephone's browser is redirected by the network to the system's Mobile website instead of the browser's normal home page. This occurs when a User accesses the Applicants network but the user can access the same Web page from other carrier's networks by directing their Web browser to the correct page.
3. The system's mobile web site provides links for the user to download the access point application to the cellular telephone.
4. The application links provided to the user can be specific to the user so that a customized version of the application can be delivered to each user if necessary.
5. The user downloads and installs the application by selecting a link from the download page.

Similarly, enhancements to the software application on the cellular telephone can be enabled in several ways:

1. The software application on the cellular telephone is signed with the permissions necessary to allow it to access persistent memory, the Bluetooth (or other short range wireless radio) subsystem on the cellular telephone and the data network. This provides the user with a better experience since the user is not prompted to allow the software application to access restricted functions on the cellular telephone.
2. The cellular telephone parses the readings to determine readings type and verify the accuracy of the reading. The software application also parses the reading in order to ask applicable lifestyle questions. However, the raw reading is transmitted to the server along with additional information from the cellular telephone so that no information is lost from the medical data reading itself.
3. The cellular telephone application is multithreaded to ensure the phone remains responsive to take calls, the Bluetooth (or other short range wireless radio) system is responsive to readings, and the GUI (graphic user interface) is responsive to new input while the communications thread handles communications with the data centre.
4. The software application is designed to be automatically started by incoming Bluetooth (or other short range wireless radio) connections on cellular telephones that support such functionality. This removes the need for the user to need to start the application prior to taking readings.

Application Server—Data Reception and SQL Insert

As described above and shown in FIG. 8, the application server on the central system includes a “Data Reception and SQL Insert” service that places meter readings and lifestyle information into the database. As shown in FIG. 9, the method for this service generally proceeds as follows:

1) The application server accept HTTPS POST at step 902.
2) The POST is authenticated at step 904 using username/password to ensure that a valid device or client is supplying data.
3) The POST (the POST is in XML format) is parsed at step 906.
4) The meter type is then checked at step 908. For each meter type steps 910 to 926 are performed.
5) The meter data is parsed based on meter specification at step 910.
6) Retrieve alert levels from database using SQL at step 912.
7) Meter data with alert levels are compared at step 914.
8) New readings into the central database using SQL at step 916.
9) If question responses exist then store them in the central database at step 918.
10) Alerts are inserted into the central database if required at step 920.
11) Other types of alerts are triggered if necessary at 922.
12) Time of update is stored into the central database using SQL at 924.
13) If the data was submitted by a landline (POTS) system, the RADIUS server logs are queried and the calling line ID is stored in the database so that the IVR can call the user to ask lifestyle questions at step 926.
14) If there was more than one device, YES at step 928, steps 910 to 926 are repeated for each device. If there are no more devices, NO at step 928 confirmation is provided to the sender at step 930 whether data was accepted or refused.

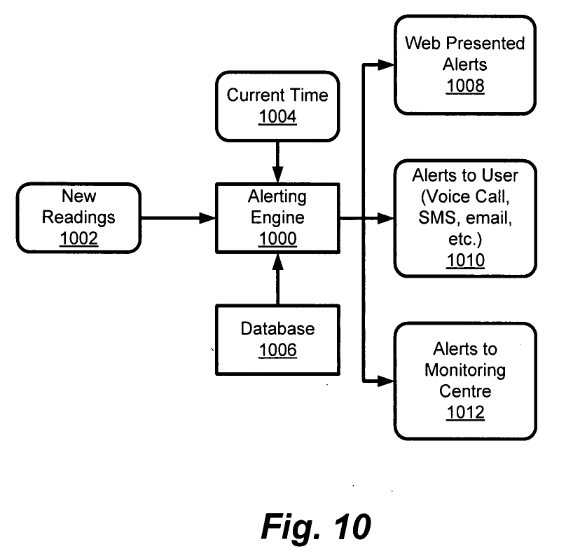

FIG. 10 presents a block diagram of an alerting engine 1000. The alerting engine is triggered based upon receiving new readings 1002 from a user, triggering events that require identification to users or Caregivers. Triggers in the database 1006 based upon the current time 1004 enable generation of alerts. The alerts can be generated by web based presentation 1008, by messaging such as voice call, SMS messages, email, etc. 1010 or by generating alerts to the monitoring centre 1012.

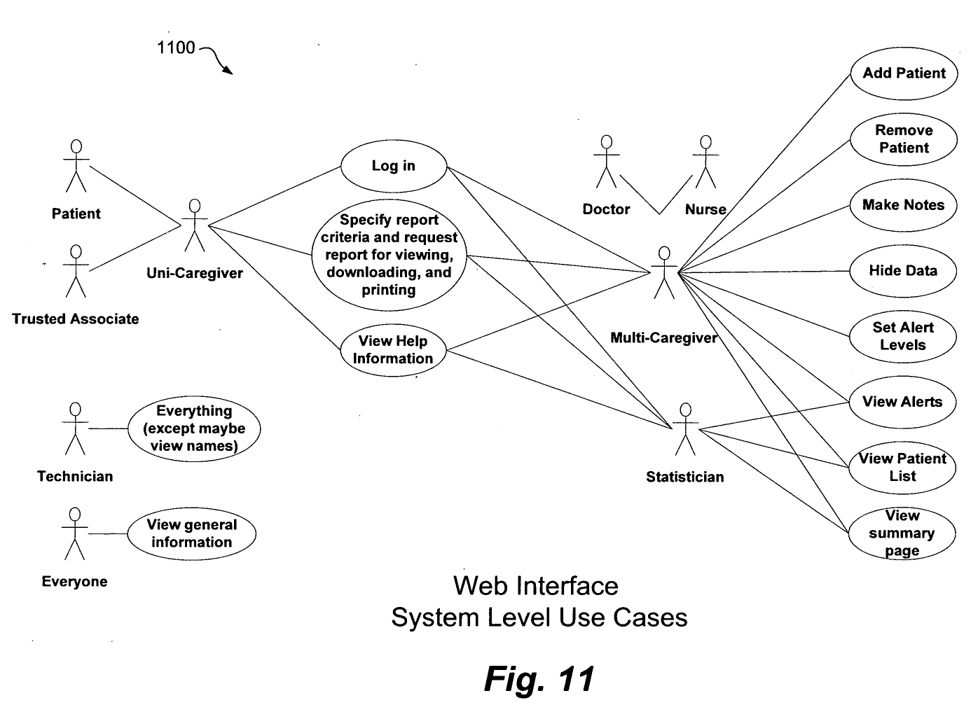

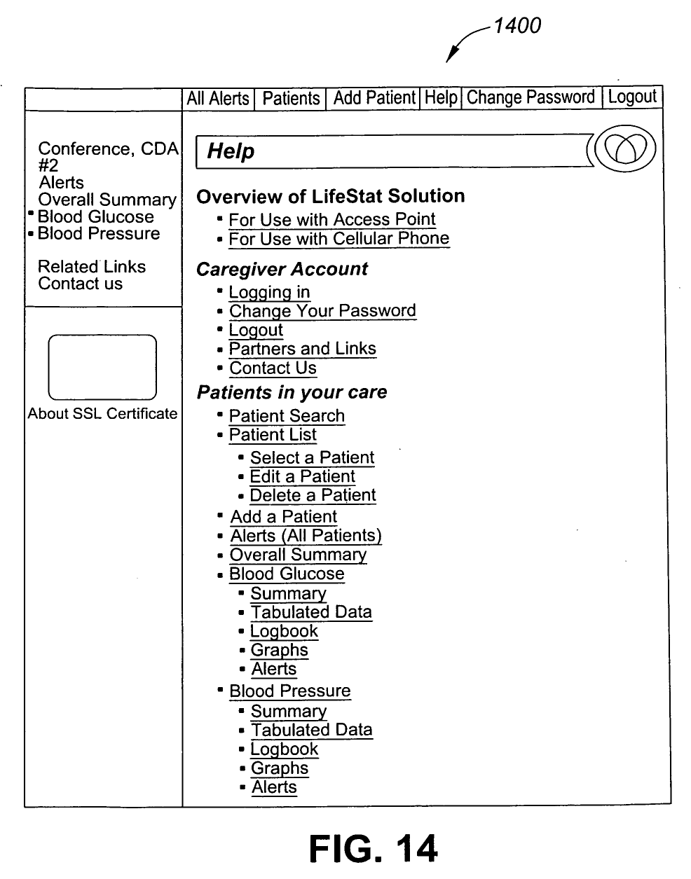

FIG. 11 presents a web interface structure 1100 showing system level use cases generated by the data centre in connection with FIGS. 14 to 36. A variety of exemplary individuals are presented in this figure, along with the extent to which they can view and/or modify data through the generated HTML web pages. One could of course, add other parties to this model or modify the accessibility rights of any party. Patient or trusted associates typically interact with a uni-Caregiver who has access to the system. The uni-Caregiver can also specify report criteria and request reports for viewing, downloading and printing. In addition, the uni-Caregiver can view help information. At the next level, the multi-Caregiver may interact with doctors or nurses or persons directed by them, using his authority to configure and access the system. The multi-Caregiver has access to functionality such as adding a patient, removing a patient, making notes, hiding data, setting alert levels, viewing alert levels, view patient list, or view summary page. A statistician can also be provided with limited access to view alerts, view patient list and view summary pages. A technician may also be given access to configure the system, though the technician may not be privy to patient identification related information. The system may also be configured to provide general information viewable to all users of the system.

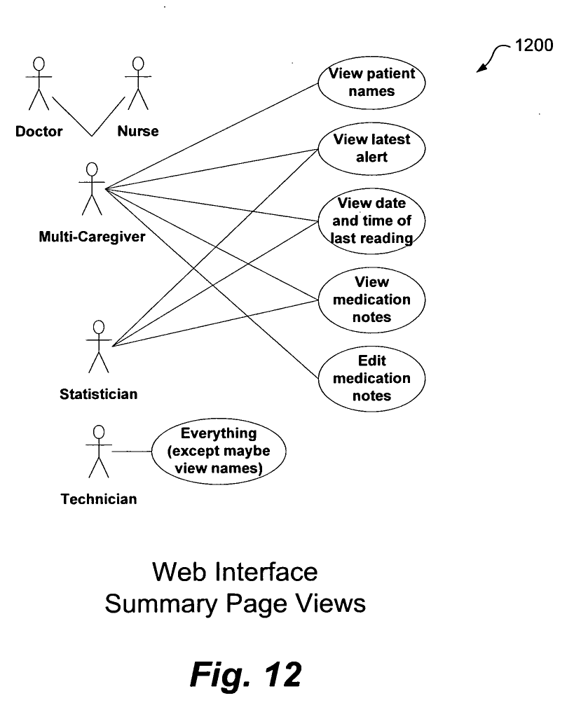

FIG. 12 presents a web interface structure 1200 identifying view summary page use cases that are only presented to those individuals who might require access to data from more than one individual, along with the extent to which they can view and/or modify data in connection with FIGS. 14 to 36. Again, the multi-Caregiver, being a doctor or a nurse or a person under the direction of a doctor or nurse can view patient names, view latest alert, view date and time of last reading, view medication notes and edit medication notes. The statistician can be provided access to limited access to view latest alert, view date and time of last reading, and view medication notes. The technician can access to configure the system but not have visibility to patient data.

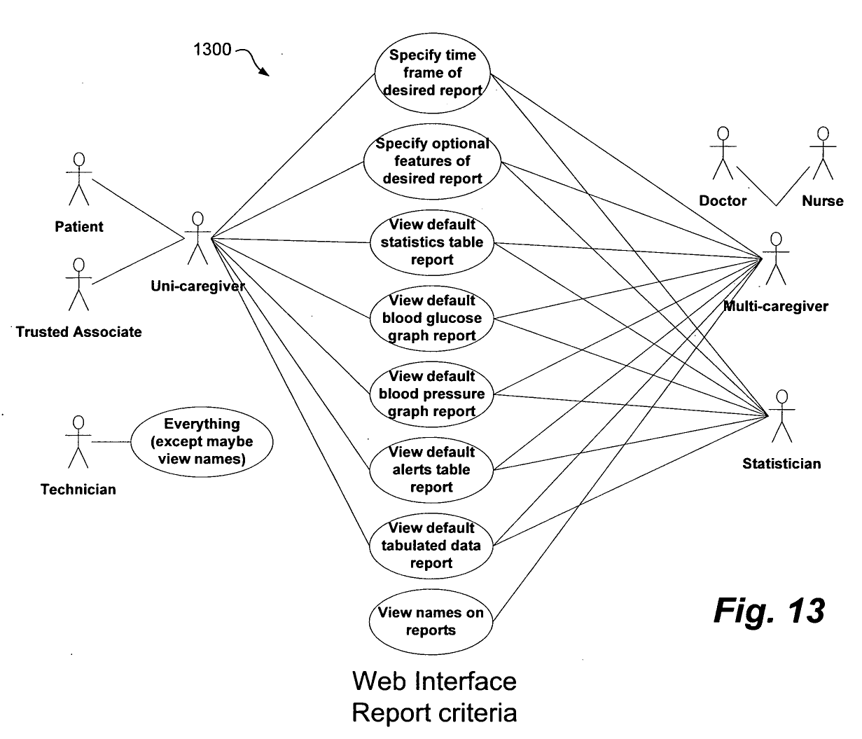

FIG. 13 presents a web interface structure 1300 identifying reporting criteria and request report for viewing, downloading and printing use cases generated by HTML web pages as described in connection with FIGS. 14 to 36. This figure presents the various reports that are available to different individuals in the system. The uni-Caregiver, multi-Caregiver and statistician can be given access to, specify time frame of desired report, specify optional features of desired report, view default statistics table report, view default blood glucose graph report, view default blood pressure graph report, view default alerts table report, view default alerts table report and view default tabulated data report. The multi-Caregiver can also be provided access to the patient names report, which the uni-Caregiver and statistician would not be given access to this information.

As noted above, the FIGS. 14 through 36 present exemplary screen captures of various graphic user interfaces (GUIs), announcements and reports generated by the system. Typically, these pages are presented to the user via HTML in a web browser but may also be implemented in a software application, or in some other manner.

FIG. 14 presents a screen capture of the main page for a Caregiver GUI 1400. The selections available to the Caregiver are organized into the following three groups:

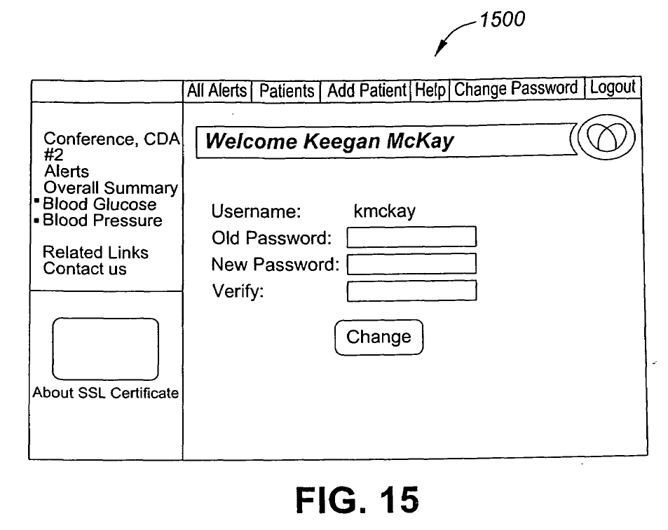

1) an overview of the system, which includes links to pages with a description of the “Access Point” (i.e. personal computer) and cellular telephone access systems;
2) personal account management tools such as logging on, changing your password, logging out, identifying partners and contacting the system managers. FIG. 15 presents a GUI for effecting the “Change Your Password” process; and
3) links and tools with regard to the patients in the Caregiver's care, which includes: patient listing and searching utilities, facilities to add patients, list alerts, provide overall summaries, provide glucose data and provide blood pressure data.

Additional details with regard to selected ones of these options are described hereinafter and are presented in FIGS. 15 through 36. The functionality of the balance of these items are generally self-evident from their descriptions.

The GUI of FIG. 14 also includes the following menu selections in a horizontal tool bar at the top of the screen:

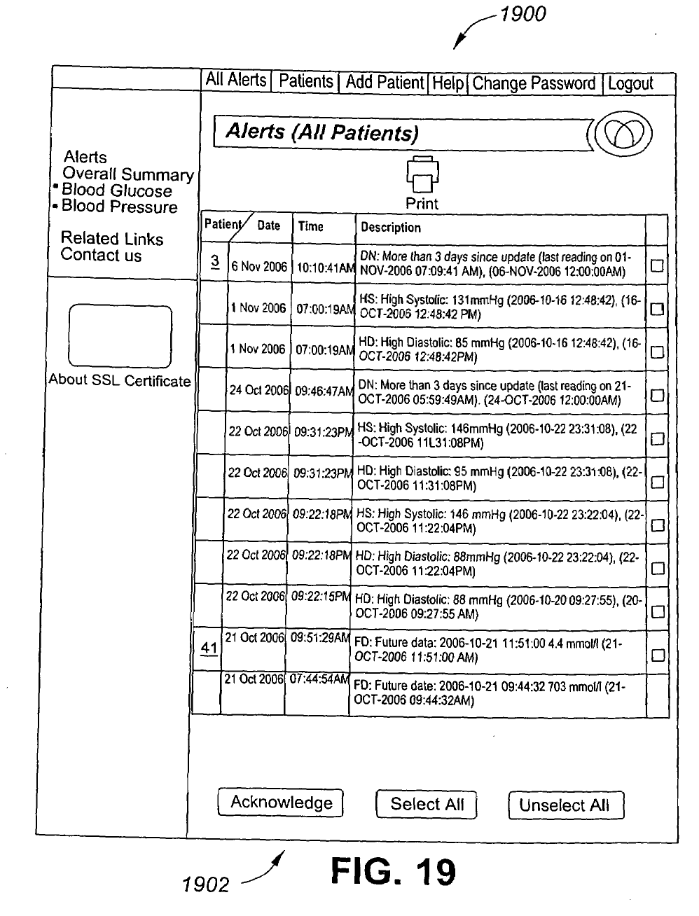

“All Alerts”—clicking on this selection presents the user with a list of all alerts that this particular user is entitled to view. If the user is a patient, this will be limited to their personal alerts, while if the user is a medical doctor, it may list the alerts for all of their patients. FIG. 19 presents an exemplary screen capture of an “all alert” report for a Caregiver with multiple patients.

The application generates timely alerts and reminders for patients to take readings or medications. In the event of a crisis situation, the patient will be contacted by a 24/7 monitoring service staffed by healthcare professionals who will recommend an immediate course of action, enabling timely interventions.

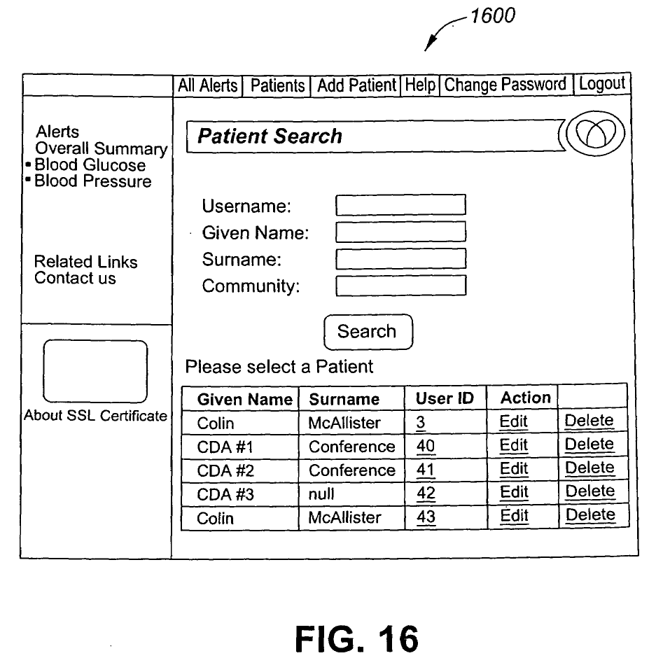

“Patients”—clicking on this selection allows the user to access the patient search page shown in FIG. 16.

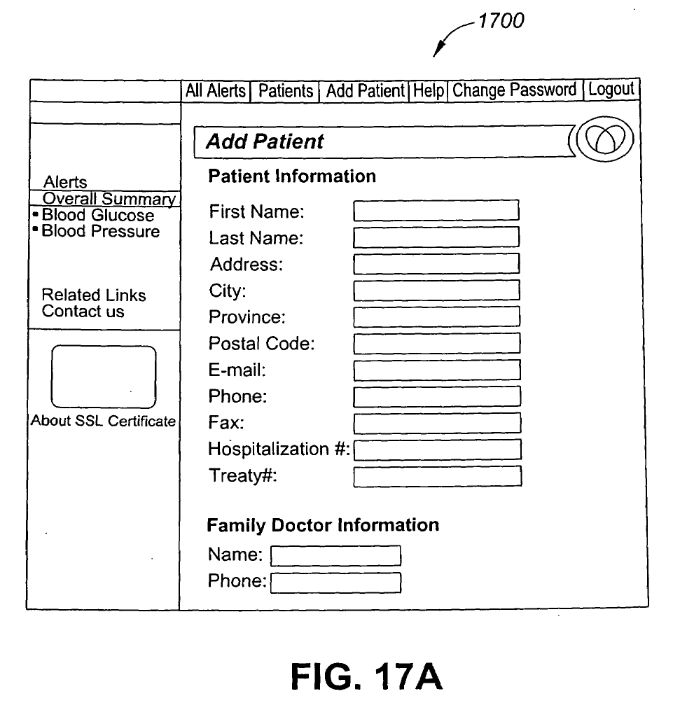

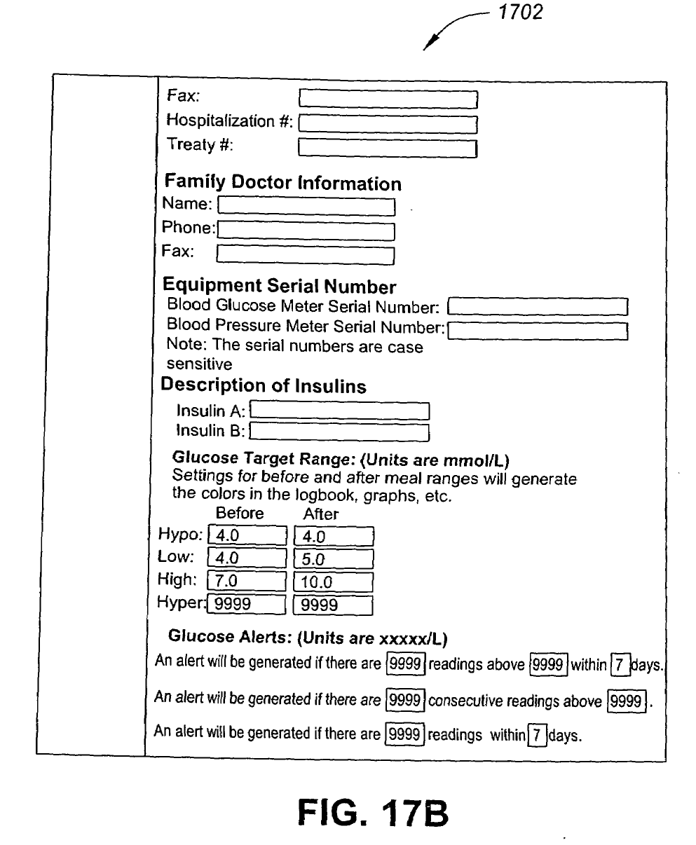

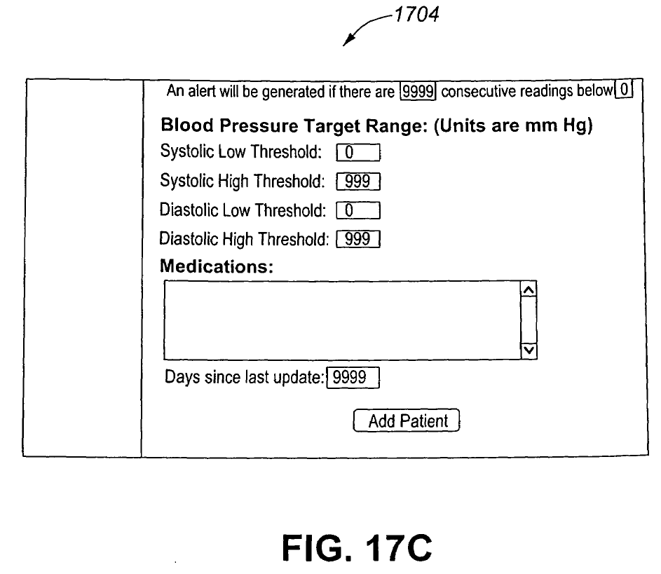

“Add Patient”—clicking on this selection allows the user to access the new patient entry page shown in FIG. 17A-C.

“Help”—clicking on this selection provides the user with access to a standard “Help” system. This could, of course, be provided in many different ways.

“Change Password” clicking on this selection allows the user to change his password.

“Logout”—clicking on this selection causes the user's account to be closed, and the browser to return to the system home page. Depending on the implementation of the system, it may also cause any data amendments made, to be compiled and archived. Given the critical nature of medical data and support systems, it is important that the system be implemented with suitable record keeping, auditing, recovery and redundancy systems.

The left hand side of this GUI also provides a number of other menu selections arranged in a column. The first entry, “Conference, CDA #2” is the name of the current or last patient whose file was considered, in “surname, given name” format. The balance of the times are menu tabs. Clicking on these menu tabs provides the following:

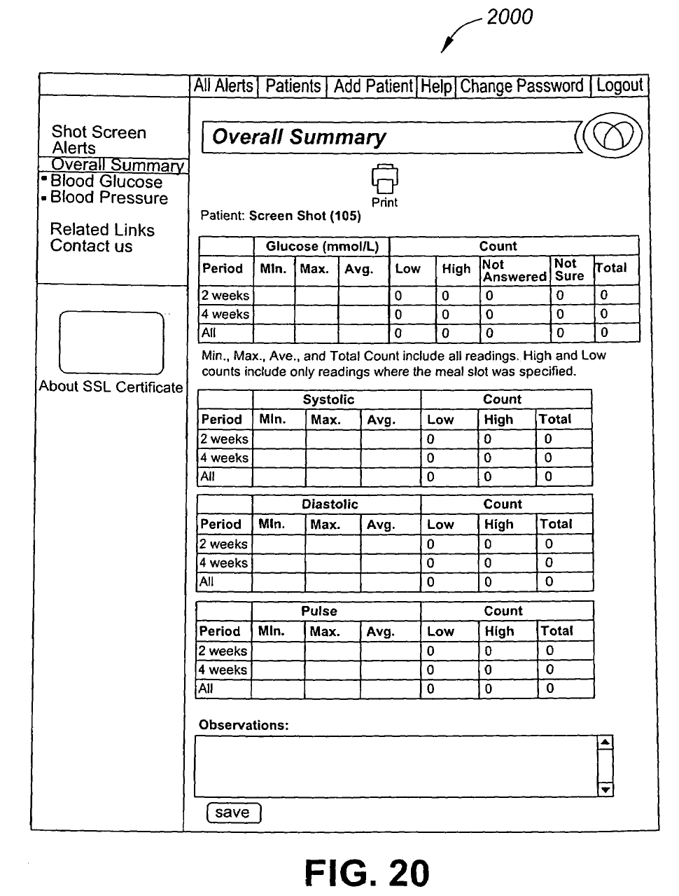

“Alerts”—lists the alerts associated with the identified patient “Overall Summary”—causes an “overall summary” report page to be presented for the particular user, as shown in FIG. 20.

“+Blood Glucose”—clicking on the “+” icon causes a listing of four menu items to be presented: “Summary”, “Tabulated Data”, “Logbook” and “Graphs”. Clicking on each of these brings up the interfaces presented in the following FIGS. (respectively): 21, 22, 24 and 25.

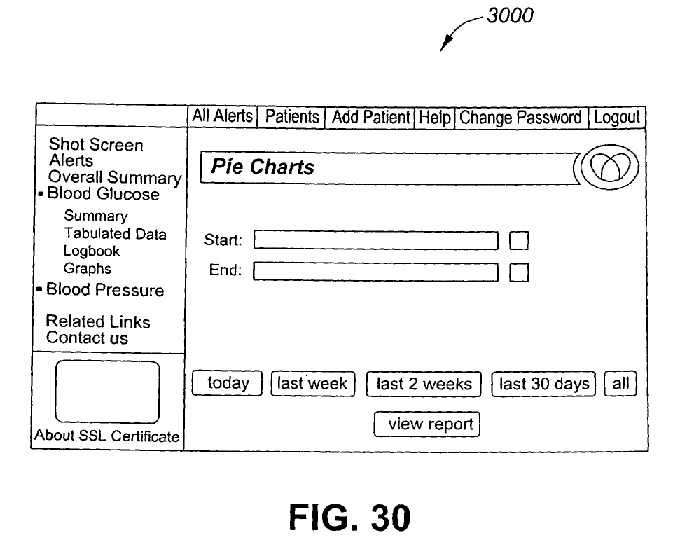

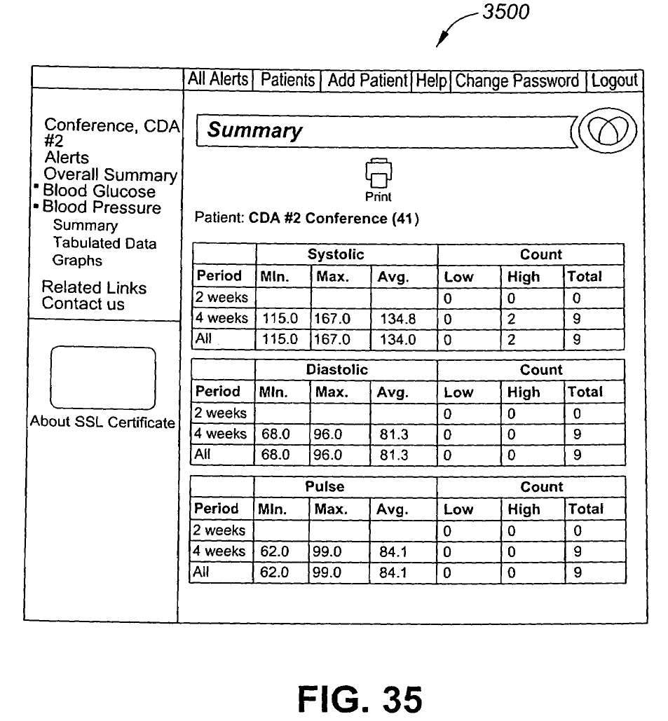

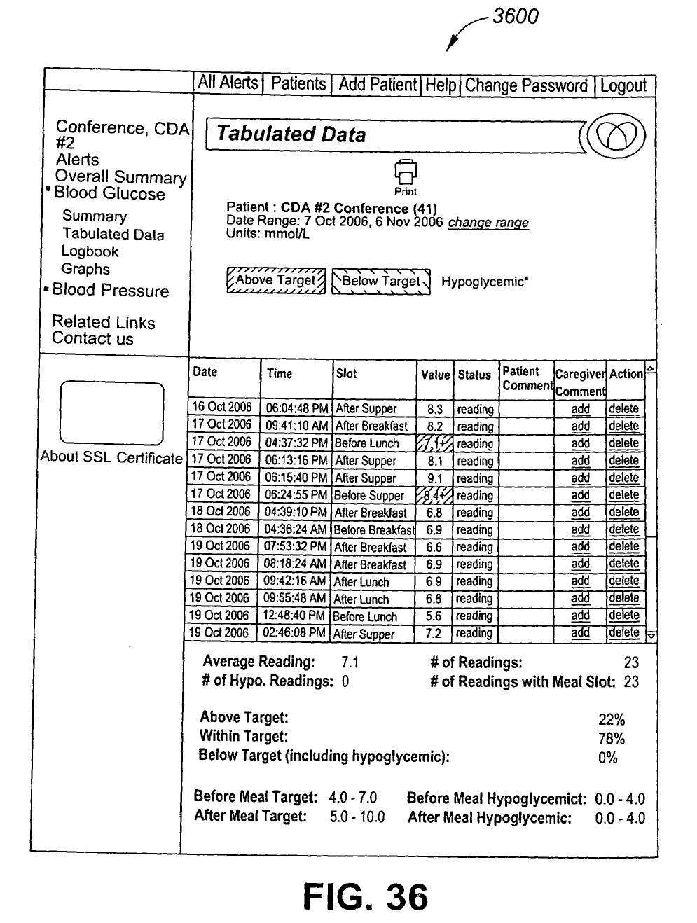

“+ Blood Pressure”—clicking on the “+” icon causes a listing of three menu items to be presented: “Summary”, “Tabulated Data” and “Graphs”. Clicking on the “Summary” and “Tabulated Data” three items brings up the interfaces presented in FIGS. 35 and 36 respectively. A figure is not included for the “Graphs” selection, but it would be effected in a manner comparable to that of the Blood Glucose FIGS. 30 to 34.

“Related Links”—clicking on this entry causes a page to be presented which may include links to sources of useful information, business partners, and related services or service providers.

“Contact Us”—clicking on this entry causes a page to be presented with standard contact information associate with the system administrator: contact name(s), address, telephone numbers, email addresses, hours for help access, etc.

Many of the other GUI and reports also include the same horizontal toolbar and menu selections in the left hand column.

If the user selects the “Change Your Password” selection in the GUI 1400 of FIG. 14, then the GUI 1500 of FIG. 15 is launched. The user must already be entered into the system in order to access this screen, so the username data is already known to the system and can be displayed. The username display is needed because some users may have multiple accounts (for example, an administrative assistant working for several Caregivers) and some computers will have multiple users: The user is then prompted to enter their old password so the system can ensure that it is the actual user who is changing the password. The user is also prompted to enter the new password twice, so the system can compare the two and confirm that the new password was correctly entered.

Selecting the “Patient Search” selection in FIG. 14 launches the GUI 1600 of FIG. 16, allowing an administrator, physician, or other Caregiver to search for a particular patient using username, given name, surname and/or community fields. The search results are presented in a table listing the given name, surname and user ID of each hit. Corresponding to each hit are action items such as “user ID”, “edit” or “delete” tabs. Clicking on the “user ID” icon for a given hit, presents the detailed user identification information for that hit. Similarly, clicking on the “edit” icon for a given hit allows the user to edit the file for that hit, while clicking on the “delete” tab deletes the record altogether.

FIG. 17A-C presents a GUI (1700, 1702, 1704) for entering a new patient onto the system. The detailed fields and labels are clear from the attached, but in short, the following groups of data are collected:

“Patient Information” such as name, address, telephone and email addresses.

“Family Doctor Information”

“Equipment Serial Number”

“Description of Insulins”

“Glucose Target Range”

“Glucose Alerts”

“Blood Pressure Target Range”

“Medications”

Once all of the data have been entered, the user clicks on the “add patient” tab, and the system will do a brief check of the data and create a data record. If unacceptable data is entered into a field, the system will bring this to the attention of the user and ask that it be corrected. As well, certain fields are identified as mandatory. If the user does not enter data into those fields, the system will prompt the user for the data.

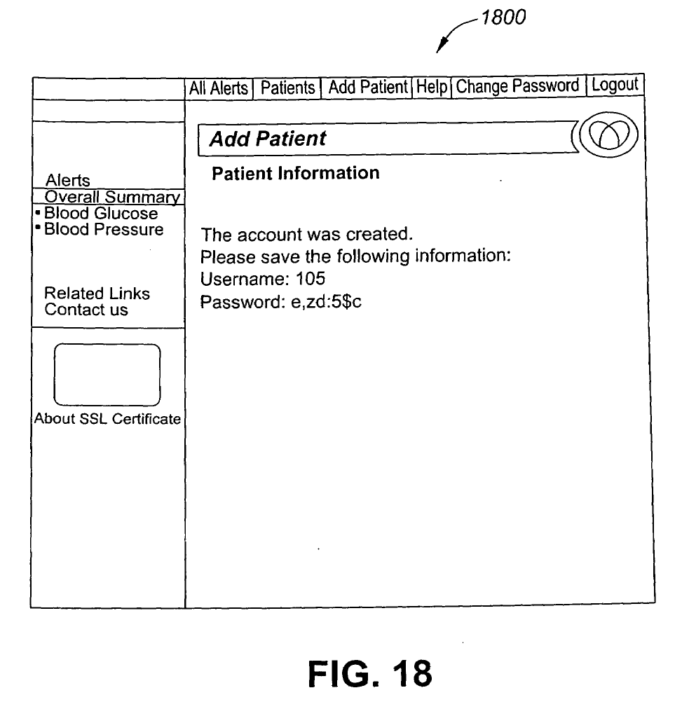

Once the new patient data has been entered and stored, a confirmation 1800 is returned to the user as shown in FIG. 18. In short, the report of FIG. 18 provides confirmation that the new record was created, and reiterates the username and password back to the user with a reminder that it should be recorded for future access.

FIG. 19 presents an “all alerts” report 1900 for a Caregiver with multiple patients. The report includes a table with columns listing patient, date of the alert, time of the alert and description of the alert. The user may click on the patient entry to link to the particular patent's main page, or to other reports/pages corresponding to that patient.

Each event also includes a tick box, allowing the user to select particular records so they can be “acknowledged” (1902) and have them removed from the report. For convenience, “select all” and “unselect all” tabs are also provided. The report may also be printed out by clicking on the “print” icon.

When the Caregiver clicks on the “Overall Summary” tab in the left column menu, the system collects and analyzes the necessary data, generating the summary report 2000 for the current patient as presented in FIG. 20. As shown, this report includes the title, patient name, summary of glucose data (minimum, maximum, average and counts for various time periods), and summary of blood pressure data (systolic, diastolic, pulse and counts, for various time periods). There is also an editable text field in this report, allowing the Caregiver to enter and save his or her observations. Physical copies of the report may be generated by clicking on the “print” icon.

As noted above, there a number of different blood glucose reports available including summary, tabulated data, logbook and graphic reports. The summary-type reports shows patient adherence as well as how often values have been out of the desired ranges. The tabulated data reports include the date and time of each reading, allowing the Caregiver to discuss specific readings with the patient. One can also quickly identify readings that fall out of recommended ranges. The electronic logbook report is designed to represent the patient's paper logbook, allowing the Caregiver to see how readings, medications, exercise, etc. relate to mealtimes. Summary statistics are typically found at the bottom of the logbook report for quick reference.

The graphic reports may use any manner of modern graphic presentation techniques, though only pie graphs are shown in these figures. The graphic report charts show a breakdown of blood sugar or blood pressure tendencies according to time of day-easily conveying trouble spots where readings fall either above or below target ranges. Choose from a variety of graphs and charts to get an overall picture of a patient's health trends.

The time ranges on these reports are typically customizable, allowing Caregivers to tailor the service particular to the patients individual situation. Color schemes generated throughout the application allow for easy identification of readings that do not meet recommended ranges.

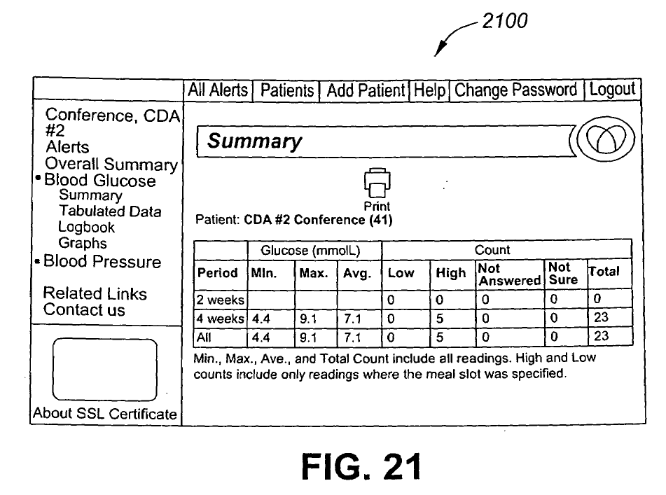

FIG. 21 presents a screen capture of an exemplary summary report 2100. Clicking on the “Summary” tab of the “Blood Glucose” menu causes the system to collect data for the current patient and generate the report as shown, which includes: title, patient name and a table with minimum, maximum, average glucose levels and counts for various time periods (two weeks, four weeks and “all”, in this example). The Caregiver may also print out hard copies of these reports by clicking on the “print” tab.

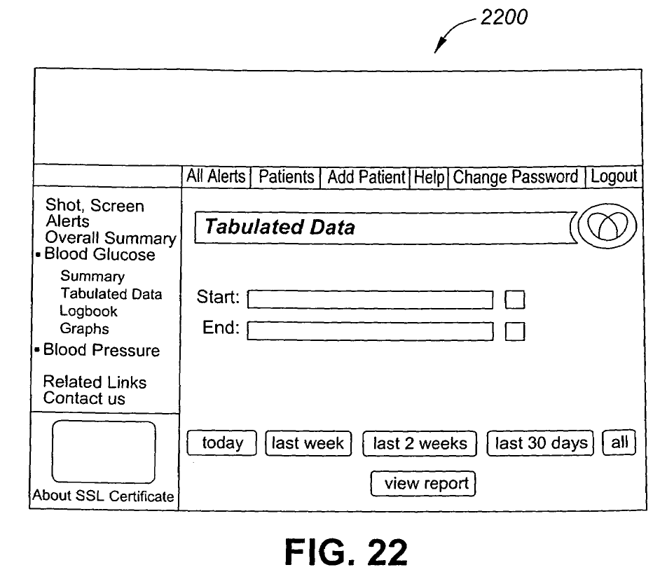

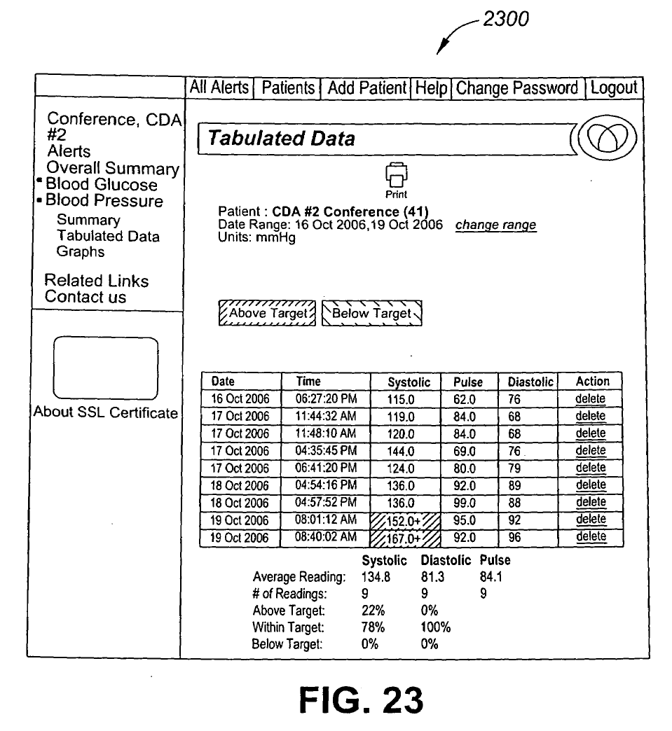

If the Caregiver clicks on the “Tabulated Data” selection of the “Blood Glucose” menu, the system returns the GUI 2200 of FIG. 22 which prompts the user for the parameters to generate a tabulated data report. As shown, the user is required to specify the start period and end period by date, using the interactive calendar utility, or by clicking on the “last week”, “last 2 weeks”, “last 30 days” or “all” tabs. Of course, other mechanisms could also be used to establish the time frame of the report. Clicking on “view report” tab causes the system to collect and compile the data, presenting it in a report 2300 as shown in FIG. 23.

The tabulated data report 2300 of FIG. 23 presents all of the glucose data entries made by the patient along with some general summary data calculated by the system. In presenting all of the data entry records to the Caregiver he or she is able to delete obviously erroneous data (via the “delete” tab associated with a given record) or to add comments (similarly, via the “edit” tab associated with a given record). Among other things the report includes: the title, a “print” icon which can be clicked on to print a hard copy, the patient name, the date range of the report, the tabulated data, the average reading, number of readings, percentage above, within and below the target values, and the data ranges before and after meals.

The data table itself includes columns for data, time, slot, value, status, patient comments, Caregiver comments and actions. Values that are above target, below target or hypoglycemic are brightly colored to contrast from other entries and the background, to attract the attention of the viewer.

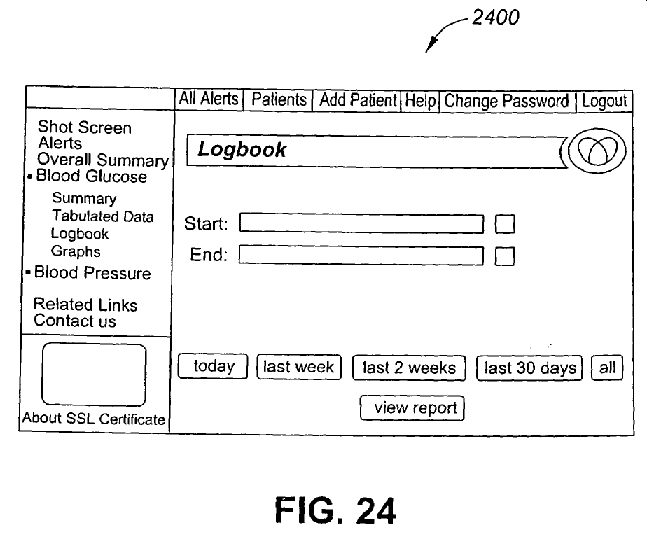

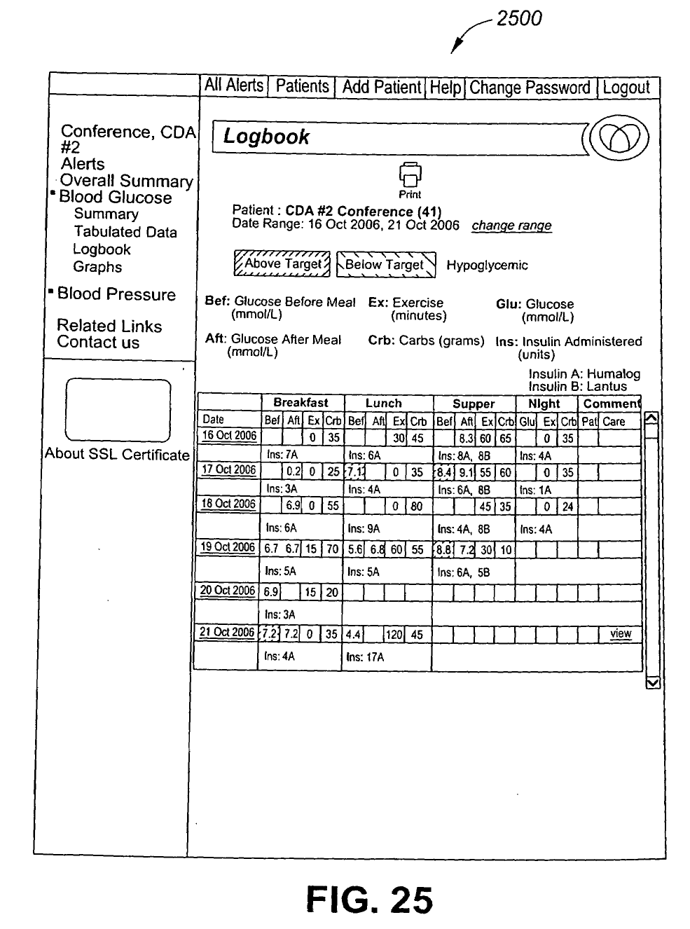

If the Caregiver clicks on the “Logbook” selection of the “Blood Glucose” menu, the system returns the GUI 2400 of FIG. 24 which prompts the user for the parameters to generate a logbook data report 2500. As shown, the user is required to specify the start period and end period by date, using the interactive calendar utility, or by clicking on the “last week”, “last 2 weeks”, “last 30 days” or “all” tabs. Of course, other mechanisms could also be used to establish the time frame of the report. Clicking on “view report” tab causes the system to collect and compile the data, generating the report as shown in FIG. 25.

The logbook data report 2500 of FIG. 25 presents all of the glucose and related data entries made by the patient. The report includes: the title, a “print” icon which can be clicked on to print a hard copy, the patient name, the date range of the report and a table of logbook data. The logbook data table itself includes columns for the date and breakfast, lunch, supper and night entries, as well as a comment column. For each of the breakfast, lunch and supper entries the patient is able to enter the mmol/L of glucose taken before and after the meal, the amount of exercise, grams of carbohydrates, and insulin administered. In the case of the “night” column, the patient is able to enter the glucose taken, exercise in minutes and carbohydrates taken.

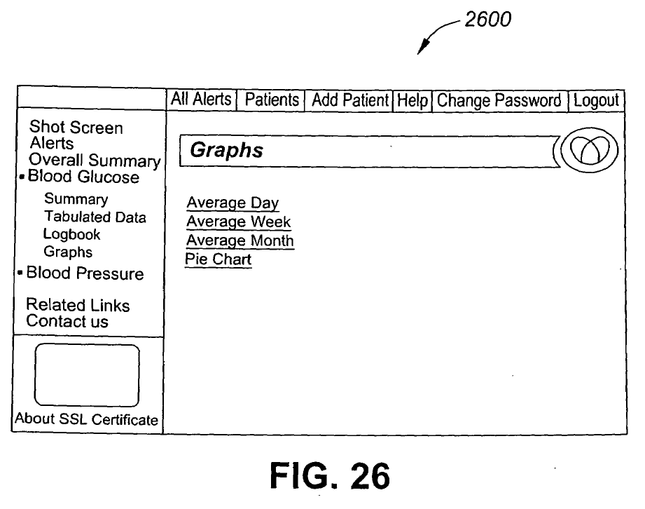

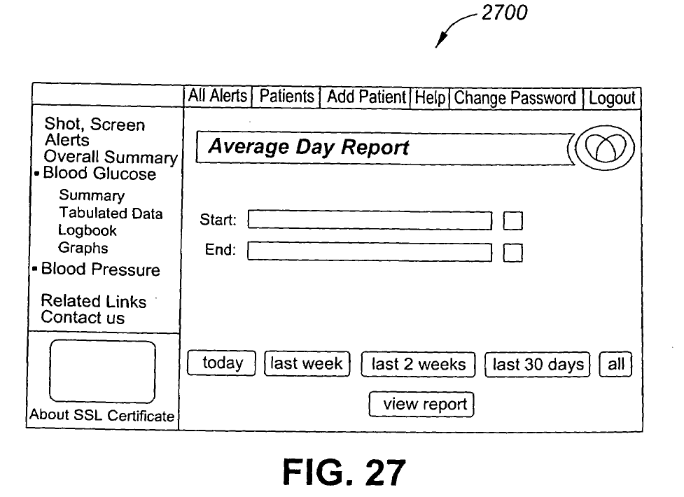

Clicking on the “Graphs” tab in the “Blood Glucose” menu returns the GUI 2600 shown in FIG. 26. Clicking on the “average day”, “average week”, “average month” and “Pie Charts” selections, sends the user to the corresponding GUIs (2700, 2800, 2900, 3000); respectively, FIGS. 27, 28, 29 and 30.

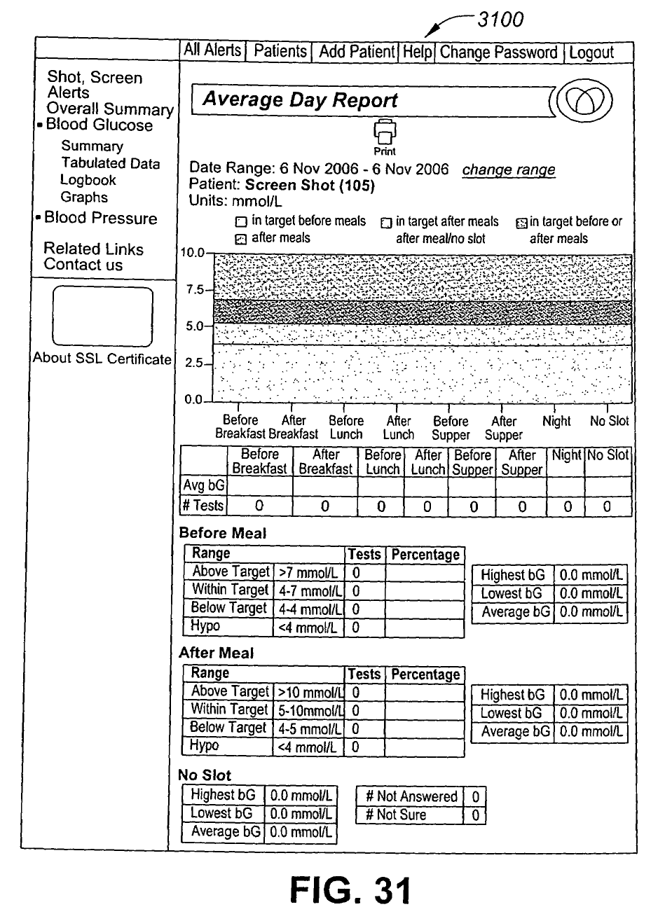

FIG. 27 presents a GUI 2700 for specifying the blood glucose average day reports. The user simply specifies the start period and end period by date, using the interactive calendar utility, or by clicking on the “last week”, “last 2 weeks”, “last 30 days” or “all” tabs. Other selection mechanisms could also be used. Clicking on “view report” tab causes the system to collect and compile the data, performing calculations and generating the report 3100 shown in FIG. 31.

The blood glucose average day report 3100 of FIG. 31 includes the title of the report, “Average Day Report”, the date range, patient name, a graphic presentation of the analyzed data (a line diagram of the blood glucose readings through the course of the average day), and calculated weekly average data. The data are organized, as shown, into before and after meal groupings, and are averaged by day. This allows the user or Caregiver to identify trends that occur on a daily basis that may have a negative impact on the blood glucose levels. This could be, for example, the regular habit of having a meal that is too large, too small, skipping breakfast periodically, having poor snacking habits, etc.

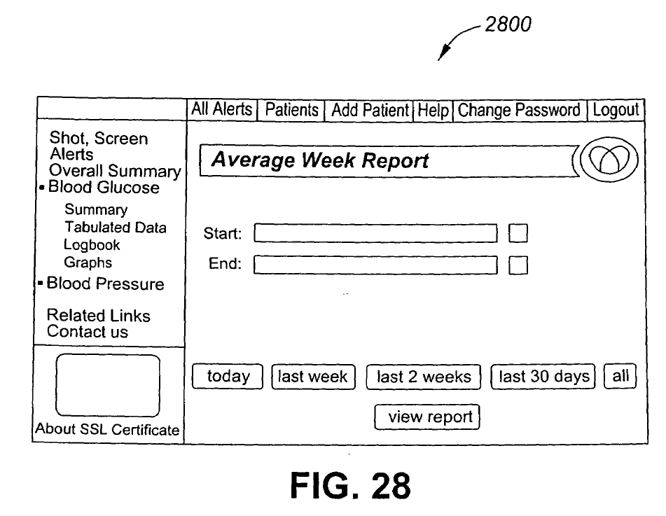

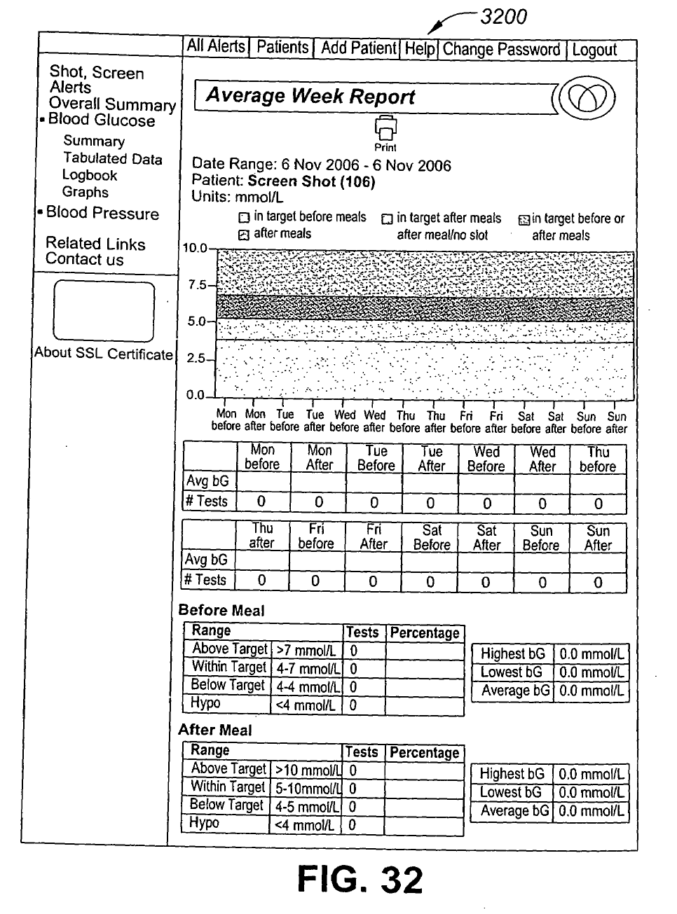

FIG. 28 presents a GUI 2800 for specifying the requirements for a blood glucose average week report. The user specifies the start period and end period by date, using the interactive calendar utility, or by clicking on the “last week”, “last 2 weeks”, “last 30 days” or “all” tabs. Other selection mechanisms could also be used. Clicking on “view report” tab causes the system to collect and compile the data, presenting it in a report 3200 as shown in FIG. 32.

The blood glucose average week report 3200 of FIG. 32 includes the title of the report, “Average Week Report”, the date range, patent name, a graphic presentation of the analyzed data, and calculated weekly average data. The data are organized, as shown, into before and after meal groupings, and averaged by week. This allows the user or Caregiver to identify trends that may have a negative impact on the blood glucose levels, such as the regular habit of a particular physical activity, Sunday dinner, sleeping in on Saturday morning and missing breakfast, etc.

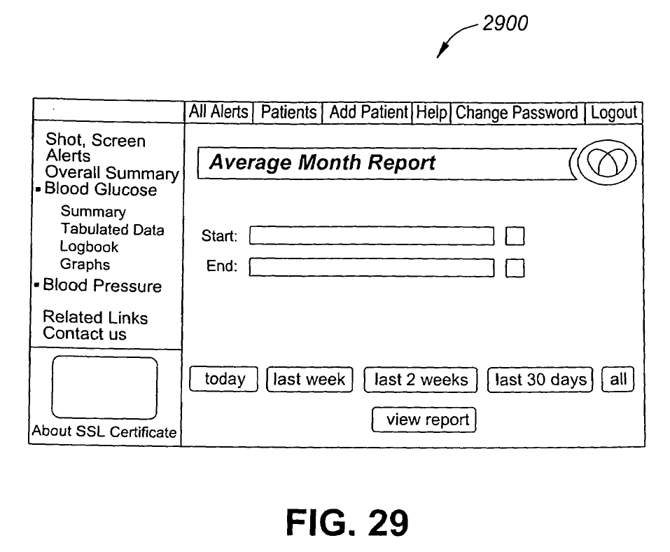

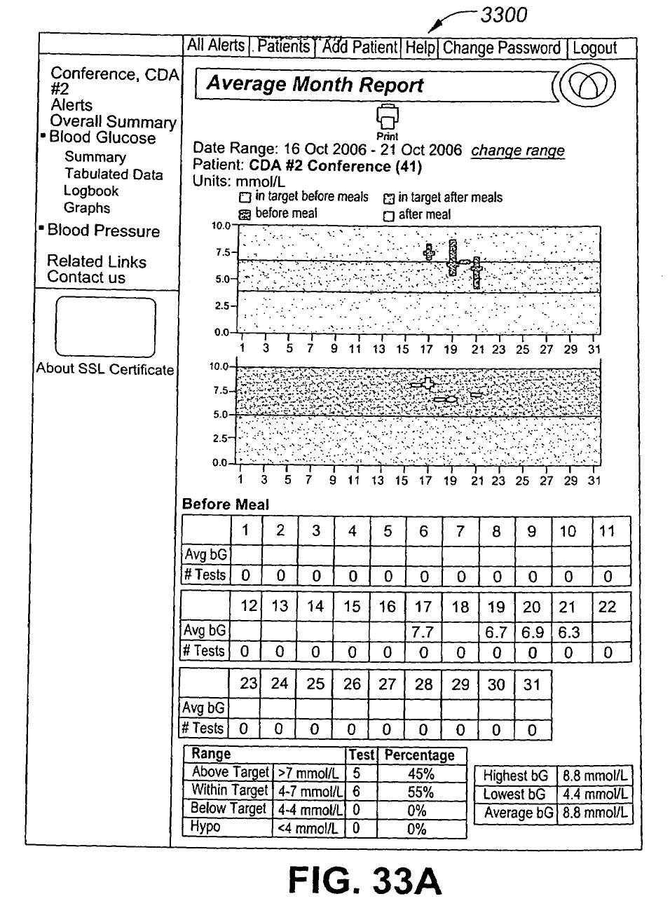

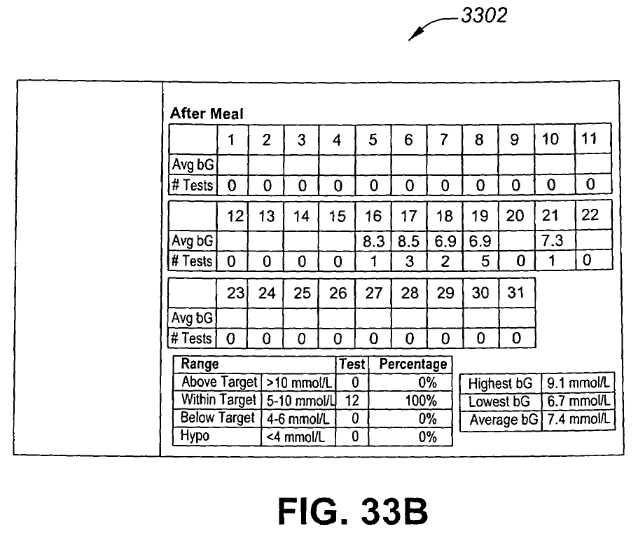

Similarly, FIG. 29 presents a GUI 2900 for specifying the requirements for a blood glucose average month report, which is intended to show trends that occur over the course of each month. Again, the user specifies the start period and end period by date, using the interactive calendar utility, by clicking on the “last week”, “last 2 weeks”, “last 30 days” or “all” tabs, or using some other selection mechanism. Clicking on the “view report” tab causes the system to collect and compile the data, analyzing it and presenting it in a report as shown in FIG. 33A-B.

The blood glucose average month report, 3300 & 3302, of FIG. 33A-B includes the title of the report, “Average Month Report”, the date range, patient name, graphic presentation of the analyzed data, and calculated month average data. The graphic presentation may show the average and range for each data point as shown, or be in some other format. The data are organized, as shown, into before and after meal groupings, and averaged by the day of the month. This allows the user or Caregiver to identify trends that may have a negative impact on the blood glucose levels, which occur over the course of each month.

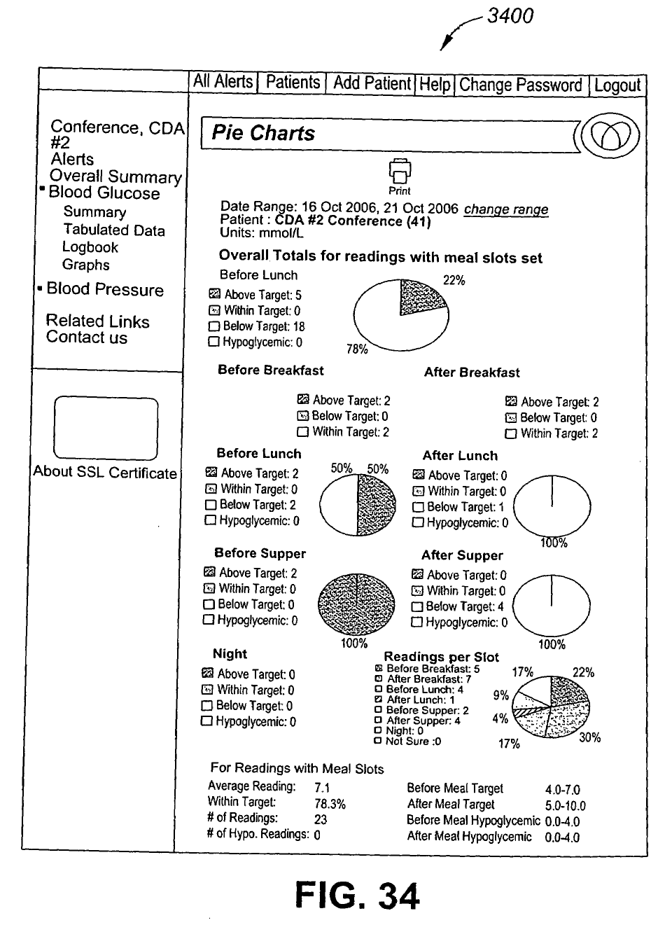

FIG. 30 presents a GUI 3000 for specifying the requirements for a blood glucose graphic report. In this embodiment, pie charts are being used, but other graphic presentations could also be used. The user specifies the start period and end period by date, using the interactive calendar utility, or by clicking on the “last week”, “last 2 weeks”, “last 30 days” or “all” tabs. Of course, other selection mechanisms could also be used. Clicking on the “view report” tab causes the system to collect and compile the data, analyzing it and generating the report 3400 shown in FIG. 34.

The blood glucose graphic report 3400 of FIG. 34 includes the title of the report, “Pie Charts”, the date range, patient name, graphic presentations of the analyzed data (in pie charts, of course), and a set of summary data calculated by the system. In this example, pie charts were generated presenting the percentages of glucose readings that were above target, below target, within target and hypoglycemic. The time periods presented are before lunch, after lunch, before supper, after supper and at night. A pie chart is also presented showing the number of readings per time slot. Of course, other time periods, data and presentations could also be used.

Clicking on the “Summary” tab of the “Blood Pressure” menu causes the system to collect the blood pressure data for the current patient, perform the necessary calculations and generate the report 3500 of FIG. 35. This report presents a summary table of blood pressure test data for a given patient, including the minimum, maximum and average values and number of count times, for each of: systolic pressure, diastolic pressure and pulse rate.

Similarly, clicking on the “Tabulated Data” tab of the “Blood Pressure” menu causes the system to collect the blood pressure data for the current patient, perform the necessary calculations and generate the report 3600 of FIG. 36. This figure presents a “Tabulated Data” report of blood pressure readings for a given patient, listing the date, time, systolic and diastolic pressures, and pulse rate for each test. Values which are “above target” or “below target” are identified with a colored box which contrasts from the balance of the screen, drawing the user's attention. The date range may be changed by clicking on a “change range” link. Data points may also be deleted.

Summary data are also calculated and presented at the bottom of the report—i.e. the average, number of readings, % above target, % within target and % below target, for each of: systolic pressure, diastolic pressure and pulse rate.

Options and Alternatives

While particular embodiments of the present invention have been shown and described, it is clear that changes and modifications may be made to such embodiments without departing from the true scope and spirit of the invention.

The requirements of healthcare professionals and patents vary greatly from one application to the next. As a result, many different user interfaces, functions and health lifestyle and wellness parameters may be required. The invention can support all of these variations, but it is impossible to outline them herein in their entirety. For example:

1) Medication record integration: The system provides the ability to track medication. This may be integrated with compliance monitoring. The system will facilitate alerting for users and Caregivers about missed doses. The system will also be able to report not only on prescribed dosage but on administered dosage and adherence to scheduling.
2) Correlations: The system provides the ability to visualize how medications and physiological parameters interact with each other. This facilitates improved diagnosis of patient response to drugs and lifestyle variations. Furthermore, the physiological measurement may be correlated against actual dosage alongside prescribed dosage. It also provides reinforcement to users about positive lifestyle actions and training material for Caregivers when educating patients.
3) Advanced Reminder Scheduling: The system provides incentive based compliance reminders based on a predetermined schedule. The scheduling may be enhanced by reacting to alert conditions such as sending reminders “only when a medication dose or scheduled reading is missed”.
4) Lifestyle monitors: The system may extend into lifestyle monitoring including activity and food monitoring. The system may include many devices not considered medical in nature such as pedometers, motion detectors, exercise equipment monitors, etc. These provide feedback to the user to correlate health indicators with activity and other lifestyle gauges;
5) the system may be integrated with other medical information systems, allowing the data to be used to monitor drug performance, HMO's to manage their costs;
6) the system may be integrated with online email systems such as Hotmail, allowing access to all of the user's online address books;
7) the system can easily accommodate different protocols such as Zigbee and the like;
8) the system can monitor and measure a limitless variety of devices and physiological parameters, including SpO2, heart rate, video, sounds, and the like;
9) the system can interact with various devices such as set top boxes (STBs) for satellite, cable and IPTV. The STB can be used as a device to collect the information from the user in the home, the TV acting as a user interface that presents lifestyle questions. These questions would be answered using the remote control, wireless keyboard or voice commands.

Various changes and alternatives to the access point could also be implemented, for example:

1) it could be modified to provide Ethernet or WiFi (IEEE 802.11) connectivity to the Internet instead of a dial-up modem;
2) rather than the current hardware based access point, one could use a PC with a USB Bluetooth key and a downloadable software application that can provide an interactive user interface for providing status to the user and asking lifestyle questions. Such an approach is inexpensive and the Bluetooth key is very portable—it fits on keychain or in a pocket, and the Internet may be accessed from almost anywhere in the world;
3) a variation on this software access point would be to replace the downloadable application with an executable object embedded in a Web page that would cause the PC to operate as an access point when the Web page is open;
4) a variation would be to employ a combination memory and Bluetooth USB key that can contain a software access point that will launch when the key is inserted into a computer;
5) one could implement an access point with a user interface that can directly ask the user lifestyle questions; and
6) one could use an SMS or IP based messaging technique that will send lifestyle questions to any cell phone that supports SMS messaging or internet access.

### CONCLUSIONS

The present invention has been described with regard to one or more embodiments. However, it will be apparent to persons skilled in the art that a number of variations and modifications can be made without departing from the scope of the invention as defined in the claims.

The method steps of the invention may be embodied in sets of executable machine code stored in a variety of formats such as object code or source code. Such code is described generically herein as programming code, or a computer program for simplification. Clearly, the executable machine code or portions of the code may be integrated with the code of other programs, implemented as subroutines, plug-ins, add-ons, software agents, by external program calls, in firmware or by other techniques as known in the art.

The embodiments of the invention may be executed by a computer processor or similar device programmed in the manner of method steps, or may be executed by an electronic system which is provided with means for executing these steps. Similarly, an electronic memory medium such computer diskettes, CD-ROMs, Random Access Memory (RAM), Read Only Memory (ROM) or similar computer software storage media known in the art, may be programmed to execute such method steps. As well, electronic signals representing these method steps may also be transmitted via a communication network.

The system provides more efficient comprehensive care, and assisting in the management of chronic illnesses such as diabetes and hypertension. Broadly, the system provides a remote patient monitoring service that enables continuous feedback between patients and their Caregivers. The result is more efficient, comprehensive care that can reduce and avoid chronic illness related complications that lead to disability and further follow-up care.

Physiological data and answers to lifestyle questions are collected, stored, analyzed and expanded upon.

Data is accessible so one can view a patients health status no matter where you are. If a patients data indicates the need for immediate action, an alert is generated and transmitted to a monitoring station, staffed by health care professionals 24/7, which will contact the patient to discuss the readings and provide advice. Also:

Loved ones can take a more active role in loved one's health and obtain peace of mind knowing loved ones are being monitored by a trained healthcare professional 24/7.

Patients are empowered to take an active role in self management, keeping them independent and at home; receive more comprehensive care; save time and money by seeing health care professionals less frequently; and gain peace of mind and reduced anxiety; gain enhanced overall quality of life.

Social Workers have greater insight into the patient's condition and can better help patients and families cope with their health condition.

Pharmacists can monitor effects of medications on patients and give better advice on how to manage medication regimens.

Doctors can access accurate, consistent, timely data; can make informed care decisions and interventions and provide greater quality of care for patients

Nurses can optimize clinical workflow, focus on clients who need you most and enhance support opportunities with other members of the care team.

Diabetes Educators have greater insight into the patients life and condition and can tailor your coaching specifically to the patient's circumstances.

Dieticians can monitor the correlation between physiological data (weight blood pressure, glucose readings) to specific meal and nutritional intake.

The system contributes to a healthier population who require less access to the healthcare system. The system provides economic benefits by way of cost avoidance, shortened wait lists, reduced comorbidities, fewer emergency room visits and fewer hospitalizations.

All citations are hereby incorporated by reference.

The embodiments described above are intended to be illustrative only. The scope of the invention is therefore intended to be limited solely by the scope of the appended claims.

1. A system for remotely managing the health of an individual comprising:
a) a server;
b) a remote interface for entering into the server a set of queries to be answered by the individual; and
c) a remotely programmable apparatus for interacting with the individual, the remotely programmable apparatus being in communication with the server via a communication network;
wherein the server comprises:
i) means for receiving responses to the set of queries, from the remotely programmable apparatus; and
ii) database means for storing the set of queries and the received responses to the set of queries; and
wherein the remotely programmable apparatus comprises:
i) a transceiver for receiving the set of queries from the server and for transmitting the responses to the set of queries, to the server;
ii) a user interface for presenting the set of queries to the individual and for receiving the responses to the set of queries;
iii) memory means for storing the set of queries and the responses to the set of queries; and
iv) a processor connected to the transceiver, the user interface, and the memory means, for communicating the set of queries to the individual, receiving the responses to the set of queries, and transmitting the responses to the server.

2. The system of claim 1, wherein the server comprises a web server having a web page for entry of the set of queries, and wherein the remote interface is connected to the web server via the Internet.

3. The system of claim 1, further comprising at least one monitoring device for producing measurements of a physiological condition of the individual and for transmitting the measurements to the remotely programmable apparatus, the remotely programmable apparatus further including a device interface connected to the processor for receiving the measurements from the monitoring device, the memory means including means for storing the measurements, and the transceiver including means for transmitting the measurements to the server.

4. The system of claim 1, wherein the device interface includes means for interfacing with a plurality of monitoring devices.

5. The system of claim 3, wherein the server further comprises report means for displaying the responses and the measurements on the user interface.

6. The system of claim 5, wherein said set of queries is generated by parsing and analysing said responses and said measurements.

7. The system of claim 5, wherein said server is operable to perform the steps of analyzing said physiological conditions on said server, and in response to certain conditions being satisfied, transmitting queries to said individual.

8. The system of claim 5, wherein said server is operable to perform the steps of analyzing said physiological conditions on said server, and in response to certain conditions being satisfied, transmitting alarms to said individual.

9. A method for remotely monitoring the health of an individual, the method comprising the steps of:
a) providing the individual with an apparatus having:
i) communication means for exchanging data with a server through a communication network, wherein the data includes a program executable by the apparatus to communicate queries to the individual, to receive responses to the queries, and to transmit the responses to the server;
ii) memory means for storing the program and the responses to the queries;
iii) user interface means for communicating the queries to the individual and for receiving the responses to the queries; and
iv) processor means connected to the communication means, the user interface means, and the memory means for executing the program;
b) entering in the server the queries to be answered by the individual;
c) on the server, generating the program from the queries;
d) transmitting the program from the server to the apparatus through the communication network;
e) executing the program in the apparatus to communicate the queries to the individual, to receive the responses, and to transmit the responses to the server; and
f) receiving and storing the responses in the server.

10. The method of claim 6, wherein the server comprises a web server having a web page for entry of the queries, and wherein the queries are entered by accessing the web page through the Internet and entering the queries in the web page.

11. The method of claim 6, wherein the apparatus further comprises a device interface connected to the processor means for receiving from a monitoring device measurements of a physiological condition of the individual, and wherein the method further comprises the steps of:
a) collecting the measurements in the apparatus through the device interface;
b) transmitting the measurements from the apparatus to the server; and
c) receiving and storing the measurements in the server.

12. The method of claim 6, wherein said server is operable to perform the steps of analyzing said physiological conditions on said server, and in response to certain conditions being satisfied, transmitting queries to said individual.

13. The method of claim 6, wherein said server is operable to perform the steps of analyzing said physiological conditions on said server, and in response to certain conditions being satisfied, transmitting alarms to said individual.

14. A system for remotely managing the health of an individual, comprising:
a) a server;
b) an interface for programming said server; and
c) a remote device for interfacing with the individual, said remote device being operable to monitor two or more physiological conditions of said individual and communicate said physiological conditions to said server;
d) said server being operable to analyze said physiological conditions, generate feedback and transmit said feedback to said remote device.

15. A system for remotely managing the health of an individual, comprising:
a) a server;
b) a remote device; and
c) a communication network for interconnecting said server and said remote device;
said remote device for interfacing with the individual, said remote device being operable to monitor two or more physiological conditions of said individual and communicate said physiological conditions to said server;
said server being operable to analyze said physiological conditions, generate feedback and make said feedback available to said user and Caregivers.

16. The system of claim 15, wherein said remote device is operable to:
monitor disparate physiological data from two or more health monitoring devices.

17. The system of claim 15, wherein said server is operable to:
analyze said physiological conditions, and in response to certain conditions, transmit queries to said individual.

18. The system of claim 15, wherein said server is operable to:
analyze said physiological conditions, and in response to certain conditions being satisfied, transmitting alerts to said individual or the Caregiver of said individual.

19. The method of claim 8, further comprising the step of:
analyzing said physiological conditions on said server, generating feedback and transmitting said feedback to said remote device.

20. The method of claim 8, further comprising the step of:
analyzing said physiological conditions on said server, generating feedback and making said feedback available to said user and Caregivers.

21. The method of claim 8, further comprising the step of:
monitoring disparate physiological data from two or more health monitoring devices, using said remote device.

22. The method of claim 8, further comprising the step of:
monitoring two or more physiological conditions of said individual using said remote device and communicating said physiological conditions to said server; and
analyzing said physiological conditions on said server, and in response to certain conditions being satisfied, transmitting queries to said individual.

23. The method of claim 8, further comprising the steps of:
monitoring two or more physiological conditions of said individual using said remote device and communicating said physiological conditions to said server; and
analyzing said physiological conditions on said server, and in response to certain conditions being satisfied, transmitting alerts to said individual or the Caregiver of said individual.
